(19) United States
(12) Patent Application Publication
(10) Pub. No.: US 2004/0048919 A1
(43) Pub. Date: Mar. 11, 2004

(54) Compositions and methods for reduction of inflammatory symptoms and/or biomarkers in female subjects

(76) Inventors: Darlene M. Dreon, Menlo Park, CA (US); Stephen Dodge Phinney, Elk Grove, CA (US)

Correspondence Address: GALILEO PHARMACEUTICALS, INC., (PREVIOUSLY GALILEO LABORATORIES, INC.), 5301 PATRICK HENRY DRIVE, SANTA CLARA, CA, 95954, US

(21) Appl. No.: 10/612,118

(22) Filed: Jul. 2, 2003

Related U.S. Application Data
(60) Provisional application No. 60/393,550, filed on Jul. 2, 2002.
(60) Provisional application No. 60/461,325, filed on Apr. 8, 2003.

(51) Int. Cl. A61K 31/355; A61K 31/202

(52) U.S. Cl. 514/458; 514/560

(57) ABSTRACT

Medicaments and methods for the treatment and/or amelioration of certain inflammatory symptoms related to premenstrual syndrome (PMS), premenstrual dysphoric disorder (PMDD), perimenopause, menopause, endometriosis, post-partum depression, or administration of hormonal contraceptives are described herein. Medicaments of the invention comprise a tocopherol, an omega-3 polyunsaturated fatty acid, such as docosahexaenoic acid (DHA), or omega-9 polyunsaturated fatty acid, optionally, a flavonoid, and, optionally, a mineral, such as magnesium. Methods for treating or ameliorating such symptoms and methods for reducing elevated CRP and/or white blood cell (WBC) associated with such conditions using medicaments of the invention are also described.

### CROSS REFERENCE TO RELATED APPLICATIONS

This application claims the benefit of U.S. Provisional Application Serial No. 60/393,550 filed Jul. 2, 2002; and U.S. Provisional Application Serial No. 60/461,325, filed Apr. 8, 2003, all hereby incorporated herein in their entireties.

### TECHNICAL FIELD

The present invention relates to medicaments comprising a non-alpha tocopherol, and at least one highly unsaturated fatty acid, particularly an omega-3 polyunsaturated fatty acid, such as all-cis 4, 7, 10, 13, 16, 19-docosahexaenoic acid (DHA). Compositions of the invention may also include a flavonoid and/or magnesium. In some embodiments, the compositions comprise nutritional excipients and in other embodiments pharmaceutical excipients. The present invention also relates to medicaments and methods for the treatment and/or amelioration of inflammatory symptoms related premenstrual syndrome (PMS), premenstrual dysphoric disorder (PMDD), endometriosis, perimenopause, menopause, post-partum depression or administration of hormonal contraceptives, such as oral contraceptives, in females. The present invention also relates to methods for reducing elevated levels of biomarkers, such as white blood cell count (WBC) or C-reactive protein (CRP) associated with such conditions or oral contraceptive use. In related embodiments, the invention also relates to biomarkers for monitoring premenstrual symptoms in female subjects.

### BACKGROUND OF THE INVENTION

Approximately 75%-90% of women with regular menstrual cycles exhibit one or more symptoms associated with a period of several days prior to onset of menses, usually during the luteal phase of the menstrual cycle. These symptoms are generally referred to as premenstrual syndrome (PMS) and are thought to affect about 50 million women in the United States, up to 40% of whom exhibit symptoms so severe as to impair normal daily activity and relationships. Within the group suffering from PMS, approximately 3-5% experience symptoms so severe as to lead to temporary functional impairment and a diagnosis of premenstrual dysphoric disorder (PMDD). Yet another subset of premenstrual symptoms may occur in women at the end of their child-bearing years, just prior to menopause. During this time, women may experience an exacerbation of premenstrual symptoms, a syndrome now referred to as perimenopausal symptoms.

There is a wide range of symptoms associated with these conditions. These include both physical and/or behavioral manifestations which may vary in kind and intensity from person to person, and from condition to condition. Physical symptoms that are typically associated with PMS include dysmenorrhea, acne, bloating, breast tenderness, dizziness, fatigue, headache, hot flashes, nausea, diarrhea, constipation, heart palpitations, swelling of the hands and feet, and cramps. Affective and cognitive symptoms can be present in the form of mood swings, angry outbursts, violent tendencies, anxiety, nervousness, tension, difficulty concentrating, depression, crying easily, depression, food cravings, forgetfulness, irritability, increased appetite, mood swings, and increased emotional sensitivity.

PMDD is a more severe form of PMS. A diagnosis of PMDD (DSM-IV) is made on the basis of a patient having at least five of the following symptoms: mood swings, marked anger, irritability, tension, decreased interest in usual activities, fatigue, change in appetite, sleep problems, and physical problems such as bloating. At least one of the diagnosed symptoms must involve mood change. PMDD can be debilitating to sufferers of the disorder, resulting in lost work time and considerable physical as well as psychological stress.

Perimenopausal symptoms afflict women in the years preceding menopause, generally two to eight years before a woman's final menstrual period, ending about a year after it. These include many of the same symptoms associated with PMS, but may be more intense than those experienced in earlier child-bearing years. Specific symptoms may include cramps, hot flashes, night sweats, memory loss, sleeping problems, mood swings, anxiety, irritability, irregular menstrual periods, heavy periods, light periods, diminished libido, increased libido, vaginal dryness, frequent urination, migraines, bloating and breast tenderness. The terms “onset of menopause” and “menopause” are used interchangeably herein herein to mean the time at which a woman's menses cease and the one two several years thereafter, when menopausal symptoms are thought to be at their worst. Menopausal symptoms include some of the foregoing, including particularly hot flashes, night sweats, memory loss, vaginal dryness and the like. These are generally considered to be exacerbated at onset of menopause, and the one to several years that follow.

Endometriosis is a disorder that is thought to be caused by the migration of menstrual fragments (endometrial tissue) into the peritoneal cavity, possibly by retrograde flow out of the fimbriated end of the fallopian tubes. These tissues respond to the hormonal variations of the menstrual cycle, exacerbating the premenstrual symptoms commonly associated with PMS.

Pharmacologically active agents currently used to treat PMDD and PMS are less than ideal. Drugs such as serotonin re-uptake inhibitors (e.g., fluoxetine and sertraline (both FDA approved for PMDD), anti-inflammatory agents, anxiolytics, hormones, dopamine agonists, and diuretics are used for treatment of PMS, but cost and side effects are significant concerns. While there are a number of medicaments based on extracts or other complex mixtures of substances, an unmet need exists for an alternate, efficacious means to alleviate premenstrual symptoms, such as those that present in women suffering from PMS, PMDD, or perimenopausal syndrome, having well-defined components.

Magnesium deficiency has been postulated to be a predisposing factor to certain premenstrual symptoms (Abraham, G. E., J. Repro. Med. 28(7): 446-464, 1983). Alpha tocopherol has been described to be beneficial in treating certain types of premenstrual symptoms; however, women suffering from weight gain, swelling of extremities (edema), breast tenderness or abdominal bloating, categorized as “PMT-H,” were consistently not helped by the treatment. In some cases, such symptoms were exacerbated by the treatment. (London, R. S., et al., J. Reproductive Medicine, 32(6): 400-404, 1987; London, R. S., et al, J Am Coll Nutr. 3(4): 351-356, 1984; London, R. S., et al., J. Am. Coll. Nutr. 2: 115-122, 1983).

Women in their child-bearing years are also primary consumers of oral contraceptives. While most of the women who take these products do so for reasons related to family planning, a significant number take or choose these products for other reasons, such as irregular periods, PMS, acne, and the like. The vast majority of oral contraceptives consist of a combination of a progestin and estrogen that are administered concurrently for 21 days followed either by a 7 day pill free interval or by the administration of a placebo for 7 days in each 28 day cycle. The most important aspects of a successful oral contraceptive product are effective contraception, good cycle control (absence of spotting and breakthrough bleeding and occurrence of withdrawal bleeding), and minimal side effects.

Current combination oral contraceptive (OC) formulations, with lower doses of estrogen (<50 μg), have significantly less risk of adverse cardiovascular events than do older combined formulations with higher dose (>50 μg) estrogen (Burkman, R. T., Clinical Obstetrics and Gynecology 44(1), 62-72, 2001; Spitzer et al. Human Reproduction, 17 (9), 2307-2314, 2002). However, current generation progestins (e.g., desogestrel) appear less safe than earlier formulations (e.g., levonorgestrel) with regard to risk of venous thrombosis (Vandenbroucke, New England Journal of Medicine, 344(20), 527-35, 2001). In addition, recent studies have associated current OC use with risk for ischemic stroke (Gillum et al., Journal of the American Medical Association, 284(1), 72-78, 2000) and myocardial infarction (Rosenberg et al., Archives of Internal Medicine, 161 (8), 1065-1070, 2001), as well as impaired blood anticoagulant pathways (Tans et al., Thrombosis Haemostasis 84 (1), 15-21, 2000), increased cardiovascular reactivity (West et al., Annals of Behavioral Medicine, 23 (3), 149-157, 2001), and microalbuminuria (Monster et al., Archives of Internal Medicine, 161(16), 2000-2005, 2001). Thus, the effect of oral contraceptives on cardiovascular risk remains a concern.

Using a sensitive, non-quantitative immunoprecipitation technique capable of identifying CRP levels above the current normal range, Connell and Connell (Connell and Connell, American Journal of Obstetrics and Gynecology 110(5), 633-639, 1971) reported the presence of CRP in more than half of women using first generation combined or sequential oral contraceptives of the 1960's. In studies carried out in support of the present invention, we found an association of low dose oral contraceptive (OC) use and plasma levels of C-reactive protein, an acute phase reactant predictive of cardiovascular disease risk.

There remains a need for effective compositions and methods for treating and/or ameliorating inflammatory symptoms associated with conditions noted above. Specifically, there is a need for a safe, effective product that alleviates certain of the symptoms associated with PMS, PMDD, or perimenopause, specifically, though not limited to, mood swings, cramps, night sweats, hot flashes, swelling, bloating, breast tenderness, irritability and sleep disturbances. Further, there is a need for methods of quantifying and/or confirming diagnosis of premenstrual associated symptoms, using more objective criteria, such as measurable biomarkers.

The present invention provides medicaments for treating the symptoms of PMS, PMDD or perimenopause, or menopause, which are also useful in reducing inflammatory symptoms associated with contraceptive use, as outlined above. According to one theory, which is not intended to be limiting, such symptoms result from inflammation and/or inflammatory response associated with such conditions.

Studies carried out in support of the present invention have revealed certain biological markers of PMS, such as CRP, which may be used to confirm diagnosis of PMS, PMDD or perimenstrual syndrome and which may be altered by effective formulations of the present invention. In addition, methods of the present invention which reduce elevated levels of CRP in women using oral contraceptives may also reduce adverse side effects associated with such elevated CRP levels. The disclosures of all patents and publications cited herein are incorporated by reference in their entirety.

### SUMMARY OF THE INVENTION

The invention relates to medicaments and methods for ameliorating or reducing inflammatory symptoms related to a number of conditions that primarily affect females. More specifically, the invention relates to medicaments and methods that alleviate certain inflammatory symptoms associated with one or more of the following conditions: premenstrual syndrome (PMS), premenstrual dysphoric disorder (PMDD), perimenopause, menopause, and administration of hormonal contraceptives in a female mammalian subject. Other indications for which methods and medicaments of the invention may find use include, without limitation, endometriosis and postpartum depression.

According to one aspect, the invention includes a medicament that comprises a stoichiometric amount of a non-alpha tocopherol or tocopherol derivative composition and an omega-3 poly-unsaturated fatty acid. According to this aspect of the invention, the tocopherol or tocopherol derivative composition and the omega-3 poly-unsaturated fatty acid are present in an amount effective to reduce an inflammatory biomarker in said subject. Exemplary biomarkers include C-reactive Protein. (CRP) and white blood cell count (WBC), as described herein, as well as other inflammatory biomarkers described herein.

As described herein, the tocopherol composition portion of the medicament may comprise a mixture of tocopherols; however, according to one embodiment such mixture is no more than about 10% (w/w) alpha-tocopherol. In another embodiment, the tocopherol composition described above includes no more than about 5% alpha tocopherol. In further embodiment, the tocopherol composition described above includes no more than about 2% alpha tocopherol. Suitable tocopherol mixtures include, by way of example, a beta-tocopherol enriched tocopherol composition, a delta-tocopherol enriched tocopherol composition and a gamma-tocopherol enriched tocopherol composition.

In further embodiments,, tocopherol formulations include a gamma-tocopherol enriched tocopherol composition comprising at least about 60% gamma-tocopherol, or a gamma-tocopherol enriched tocopherol composition comprising at least about 90% gamma-tocopherol.

In another embodiment, medicaments of the invention will be composed of tocopherol derivatives; in some embodiments, such derivatives are metabolites of gamma tocopherol, beta tocopherol or delta tocopherol, as described herein. An exemplary metabolite in this regard is a natural metabolite of gamma tocopherol, described as gamma-carboxy ethyl hydroxy chroman (gamma-CEHC). Other useful tocopherol derivatives include tocotrienols.

In another embodiment, the omega-3 poly-unsaturated fatty acid is selected from the group consisting of docosahexaenoic acid (DHA), docosapentaenoic acid (DPA), eicosapentaenoic acid (EPA), eicosatetraenoic acid (ETA), octadecatetraenoic acid, (SDA), and octadecatrientoic acid (ALA). Preferably, such omega-3 poly-unsaturated fatty acids will contain less than about 10% of an omega-6 poly-unsaturated fatty acid. In a preferred embodiment the omega-3 poly-unsaturated fatty acid is DHA. In yet another preferred embodiment, the DHA containing medicament comprises a ratio of greater than 10:1 DHA:EPA. In still another embodiment, the medicament will contain a flavonoid compound as a further component of the medicament. Exemplary flavonoids include quercetin, hesperetin and a mixture of quercetin and hesperetin.

In still a further embodiment, the medicament may include a mineral component, either in combination with the tocopherol and omega-3 poly-unsaturated fatty acid components described above, or in combination with these components plus the flavonoid component. Certain mineral components are preferred, including copper, zinc, selenium, magnesium, calcium, molybdenum, manganese, chromium, iodine, iron and combinations thereof. More preferred are divalent ions such as magnesium.

In another useful embodiment, the medicament may comprise a gamma-tocopherol enriched tocopherol composition consisting of greater than about 60% gamma tocopherol, DHA, a mixture of hesperetin and quercetin, and magnesium. Certain ranges of these components are described, for example, in the foregoing formulation, a range of 100-500 mg of a gamma-tocopherol enriched tocopherol composition, 100-1500 mg DHA, 10-500 mg quercetin, 10-500 mg hesperetin, and 10-500 mg magnesium. An exemplary formulation includes 300 mg of gamma-tocopherol-enriched tocopherol composition compriseing about 60% gamma-tocopherol, about 10% alpha-tocopherol, and about 30% delta-tocopherol; about 800 mg DHA; about 33 mg quercetin; about 66 mg hesperetin; and about 100 mg magnesium.

Medicaments as described above may be administered in a number of forms, including potable solid or liquid, nutritional products, and the like; conveniently, the medicament will be administered orally in capsular or tablet form, and may be administered in a plurality of capsules or tablets.

Medicaments according to the invention, as described above, will be particularly useful in treating inflammatory symptoms are associated with PMS, PMDD, or perimenopause. Such medicaments are also useful in reducing certain inflammatory symptoms of hormonal contraceptive use, particularly oral contraceptive use. In addition, medicaments of the invention find use in treating inflammatory symptoms of endometriosis, menopause and post partum depression.

Inflammatory symptoms include, but are not limited to, acne, body fluid retention (“bloatedness”), edema, weight gain, breast tenderness, dizziness, dysmenorrhea, fatigue, headache, hot flashes, nausea, diarrhea, constipation, palpitations, swellings of appendages, swelling of breasts, angry outbursts, violent tendencies, anxiety, tension, nervousness, difficulty concentrating, crying easily, depression, food cravings (sweets, salts), forgetfulness, irritability, increased appetite, mood swings, overly sensitive, desire to be alone, abdominal cramps, and backache.

In yet a further embodiment, the invention includes a kit that includes the components of a medicament as described above, especially where the components of the medicament are present in a plurality of tablet or capsule forms packaged in separate containers. Such a kit may also include instructions for determining levels of specific inflammatory biomarkers, such as WBC and/or CRP. It may also include measurement means for determining levels of WBC and/or CRP, as described herein.

In still a further embodiment, the invention includes a medicament for ameliorating or reducing inflammatory symptoms associated with PMS, PMDD, perimenopause or concomitant hormonal contraceptive use in a female mammalian subject. Such medicaments are as described above, except that in place of the omega-3 poly-unsaturated fatty acid, they include an omega-9 poly-unsaturated fatty acid. An example of an omega-9 poly-unsaturated fatty acids is all cis 5,8,11 eicosatrienoic acid.

According to a further feature, the invention includes a method for ameliorating or reducing one or more premenstrual symptoms in a female mammalian subject experiencing such symptoms or at risk for experiencing such symptoms.

According to this embodiment, the method includes administering to the subject a medicament as described above, or herein.

According to one preferred embodiment, the inflammatory symptoms that are beneficially treated may include acne, body fluid retention (“bloatedness”), edema, weight gain, breast tenderness, dizziness, dysmenorrhea, fatigue, headache, hot flashes, nausea, diarrhea, constipation, palpitations, swellings of appendages, swelling of breasts, angry outbursts, violent tendencies, anxiety, tension, nervousness, difficulty concentrating, crying easily, depression, food cravings (sweets, salts), forgetfulness, irritability, increased appetite, mood swings, overly sensitive, desire to be alone, abdominal cramps, and backache.

According to one embodiment, the female may have one or more of these symptoms, and therefore subject to treatment, during the luteal phase of her menstrual cycle, and specifically during the late luteal phase. According to a further feature, methods and medicaments of the invention may have a beneficial effect on dysmenorrhea occurring during late luteal phase or after onset of menstruation. According to a further feature of the invention, treatment may be given during these time intervals, or across the menstrual cycle, to the benefit of the subject.

According to still yet a further embodiment, the invention includes a method of reducing body fluid retention in a female mammalian subject. This method is particularly salutary to reducing body fluid retention (“bloating” or “bloatedness”) that occurs during the luteal phase of the woman's cycle. The medicaments of the invention, as described above and herein, also provide benefit in this embodiment.

According to a further embodiment, the invention includes a method of reducing premenstrual weight gain in a female mammalian subject. In a preferred embodiment, this method is particularly applicable to reducing weight gain that occurs during the luteal phase of the woman's cycle. The medicaments of the invention, as described above and herein, also provide benefit in this embodiment of the invention.

In still another aspect, the invention includes a method of reducing the amount of analgesic and/or anti-inflammatory medication required to reduce premenstrual symptoms in a female subject. According to this aspect, a female subject suffering from, for example, PMS, PMDD or perimenopause, may find that, when administered medicaments or formulations of the invention, she will require less analgesic and/or anti-inflammatory medications (such as acetaminophen, aspirin, ibupren and the like).

In still a further, related, embodiment, the invention includes a method of measuring the effectiveness of a premenstrual intervention in a mammalian subject, comprising measuring in or from the subject a biomarker selected from the group consisting of circulating white blood cells (WBC) or C-reactive protein (CRP), wherein an effective intervention is characterized by a reduction in said circulating WBC levels and/or reduction in CRP level in the subject after intervention, compared to such levels prior to intervention (or population normalized levels). Other indicators include, without limitation, reduction in red blood cell arachidonate content, reduction in white blood cell arachidonate content, and/or reduction in mucosal cell arachidonate. Mucosal cell arachidonate may be obtained from various mucosal cells, including oral, nasal, vaginal, and rectal cells. White blood cells may polymorphonuclear leukocytes (granulocytes), mononuclear cells, lymphocytes, platelets, or eosinophils. Preferably, such measuring will be carried out during luteal phase in the subject.

These and other objects and features of the invention will become more fully apparent when the following detailed description of the invention is read in conjunction with the accompanying drawings.

### BRIEF DESCRIPTION OF THE DRAWINGS

FIG. 1 shows a comparison of the mean change in symptom scores in PMS patients given anti-inflammatory composition of the present invention or placebo over 3 menstrual cycles.

FIG. 2 shows a comparison of body fluid retention during late luteal phases over 3 cycles in women taking a composition of the invention (circles) or placebo (triangles).

FIG. 3 shows decrease in self-medication with analgesic compositions by subjects taking formulations of the invention.

FIG. 4 shows a decrease in leukocytes in subjects taking formulations of the invention.

FIG. 5 shows the increase of CRP in subjects taking oral contraceptives and non-users according to menstrual cycle phase

### DETAILED DESCRIPTION OF THE INVENTION

The present invention is directed to novel medicaments and methods for treating the physical and/or behavioral symptoms of pre-menstrual syndrome (PMS) or premenstrual dysphoric disorder (PMDD) in women with regular menstrual cycles and/or the physical and behavioral symptoms, particularly those associated with inflammation, of perimenopause or menopause. The invention also includes reducing levels of C-reactive protein (CRP) to healthy levels in women who are taking hormonal contraceptives, such as oral contraceptives. In related embodiments, the invention also includes biomarkers of PMS.

Definitions

By “amelioration” is meant improvement of the state of a subject; the amelioration of a stress is the counter-acting of the negative aspects of a stress. Amelioration includes, but does not require complete recovery or complete prevention of a stress. In the context of the present invention, amelioration is preferably at least about 30%, preferably at least about 50%, more preferably at least about 70%, even more preferably at least about 80%, and even more preferably at least about 90% reduction in the levels of a biomarker associated with premenstrual symptoms a significant reduction in one or more premenstrual symptoms, such as, for example, bloating, weight gain, or edema.

The term “medicament” means, in its broadest sense, something that treats or prevents or alleviates the symptoms of disease or condition. A medicament may be a prescription or non-prescription pharmaceutical preparation, or may also encompass a non-prescription dietary supplement, nutritional supplement or medical food having such properties.

As used herein, the term “comprising” and its cognates are used in their inclusive sense; that is, equivalent to the term “including” and its corresponding cognates.

A “contraceptive” means a drug that diminishes the likelihood of or prevents conception. A “hormonal contraceptive” is a drug that is supplements, enhances or mimics the effect of a naturally occurring female, such as estrogen or progesterone. Generally; hormonal contraceptives are ingested orally as capsules or tablets, but they may also be administered as transdermal patches or by depot injection. An “oral contraceptive” is a contraceptive, usually a hormonal contraceptive, that is taken orally. Some examples of oral contraceptives include but are not limited to combinations of various forms of estrogen and progestin, marketed in the United States under the tradenames Loestrin®, Lo/Ovral®, Nordette®, Ovcon®, Brevicon®, Demulen®, Ortho Novum®, Ovral®, Norlestrin®, Tri-Levlen®, Tri-Norilyn®; progestin alone (marketed as Micronor®, Ovrette®). Other oral contraceptives include forms marketed in the U.S. as Nordette®, Alesse®, Microgestin®, Mircette®, Ogestrel®, Triphasil®, Trivora®, and Zovia®. An exemplary transdermal patch contraceptive is the ORTHO EVRA™ (norelgestromin/ethinylestradiol transdermal system). An exemplary depot injectable composition is depot-medroxyprogesterone acetate (Depo-Provera®).

As used herein “DHA” refers to the highly unsaturated fatty acid all-cis 4, 7, 10, 13, 16, 19-docosahexaenoic acid and encompasses the free acid, methyl ester, ethylethyl ester, monoglyceride, diglyceride and triglyceride form and encompasses DHA obtainable from any source, including algal, fungal, plant, avian, fish or mammalian sources. Algal DHA is available, for example, from Martek Biosciences (Columbia, Md.) and its distributors.

The term “dysmenorrhea” refers to a uterine contractile event, in which the uterus contracts and relaxes with sufficient force to cause reduced blood supply to the uterus, reducing oxygen, and resulting in pain. Dysmenorrhea is classified as primary (spontaneous onset) or secondary (due to some inciting event). In addition to painful uterine cramping with menses, women with dysmenorrhea may experience nausea, vomiting, diarrhea, headaches, weakness, and/or fainting. Symptoms may vary in severity from cycle to cycle, but generally continue throughout the reproductive years.

By “flavonoid” is meant any of a class of polyphenolic molecules based on a flavan nucleus, comprising 15 carbon atoms, arranged in three rings as C6-C3-C6. Flavonoids are generally classified into subclasses by the state of oxidation and the substitution pattern at the C2-C3 unit. As used herein, the term “flavonoid” encompasses, but are not limited to, flavanones, flavonols, flavones, anthocyanidins, chalcones, dihydorchalcones, aurones, flavanols, dihydroflavanols, proanthocyanidins (flavan-3, 4-diols) isoflavones and neoflavones. As used herein, the term “flavonoid” encompasses, but is not limited to: diosmin, 7-[[6-O-6-Deoxy-α-L-mannopyranosyl)-β-D-glucopyranosyl]oxy]-5-hydroxy-2-(3-hydroxy-4-methoxyphenyl)4H-1-benzopyran-4-one; 3′,5,7-trihydroxy-4′methoxyflavone-7-rutinoside; 5-hydroxy-2-(3-hydroxy-4-methoxyphenyl)-7-(O6-α-L-rhamnopyranosyl)-β-D-glucopyranosyloxy)chromen-4-one; 5-hydroxy-2-(3-hydroxy-4-methoxyphenyl)-7-β-rutinosyloxy-4H-chromen-4-one; diosmetin 7-β-rutinoside; diosmine; barosmin; buchu resin; Daflon; Diosmil; Diovenor; Flebopex; Flebosmil; Flebosten; Flebotropin; Hemerven; Insuven; Tovene; Varinon; Ven-Detrex; Venex; Veno-V; or Venosmine; hesperidin, (S)-7-[[6-O-(6-Deoxy-α-L-mannopyranosyl)-β-D-glucopyranosyl]oxy]-2,3-dihydro-5-hydroxy-2-(3-hydroxy-4-methoxyphenyl)-4H-1-benzopyran-4-one; hesperetin 7-rhamnoglucoside; cirantin; hesperetin-7-rutinoside; hesperetin, (S)-2,3-dihydro-5,7-dihydroxy-2-(3-hydroxy-4-methoxyphenyl)4H-1benzopyran-4-one; 3′,5,7-trihydroxy-4′-methoxyflavanone; cyanidanon 4′-methyl ether; rutin, 3-[[6-O-(6-Deoxy-α-L-mannopyranosyl)-β-D-glucopyranosyl]oxy]-2-(3,4-dihydroxyphenyl)-5,7-dihydroxy-4H-1-benzopyran-4-one; rutoside; quercetin-3-rutinoside; 3,3′,4′,5,7-pentahydroxyflavone-e-rutinoside; melin; phytomelin; eldrin; ilixathin; sophorin; globularicitrin; paliuroside; osyritrin; osyritin; myrticolorin; violaquercitrin; Birutan; Rutabion; Rutozyd; Tanrutin.

Derivatives of diosmin are described in, for example, U.S. Pat. Nos. 5,296,469; and 4,894,449. Hesperetin can be prepared by extraction from the peel of citrus fruit or by synthesis (Shinoda et al. C.A. 23:2957, 1929; Seka et al. Monatsh. 69:284, 1936). The separation of isomers of hesperetin is described in Arthur et al., J. Chem. Soc. 632, 1956. The structure and configuration of hesperetin are described in Arakawa et al. Ann. 636:111 1960.

As used herein, the terms “inflammatory biomarkers” “biomarkers of premenstrual symptoms” or “biomarkers associated with PMS” are used interchangeably to refer to certain substances, the levels of which change in response to inflammatory events. These include, but are not limited to C-reactive protein (CRP), elevated white blood cell count (WBC), cellular arachidonic acid levels, leptins, and soluble TNF-receptors, as well as certain inflammatory markers described herein.

The terms “inflammatory symptoms related to PMS, PMDD, perimenopause, menopause or the administration of hormonal contraceptives” or “premenstrual symptoms” are further defined in the specification, and include, but are not limited to one or more of a number of symptoms commonly experienced by women in the several days prior to onset of menses (e.g., during luteal or late-luteal phase of the menstrual cycle) or during periods associated with hormonal fluctuations, as the case may be, include, but are not limited to dysmenorrhea, acne, body fluid retention (also referred to as “bloatedness” or “bloating”), breast tenderness, dizziness, fatigue, headache, hot flashes, nausea, diarrhea, constipation, heart palpitations, swelling of the hands and feet, abdominal cramps, mood swings, angry outbursts, violent tendencies, anxiety, nervousness, tension, difficulty concentrating, depression, crying easily, depression, food cravings (sweets, salts), desire to be alone, forgetfulness, irritability, increased appetite, mood swings, backache, and increased emotional sensitivity.

A “mammalian subject” includes, but is not limited to, a human or other species, such as primate monkeys, that experiences menstrual cycles.

“Omega-3 polyunsaturated fatty acids” are polyunsaturated fatty acids characterized by a methylene-interrupted structure and at least two double bonds, where the first double bond is between carbons 3 and 4, relative to the carboxyl group. The omega nomenclature describes the position of the first double bond in the hydrocarbon relative to the carboxyl alpha carbon. Omega-3 fatty acids are preferably in the natural “all-cis” configurations. Omega-3 polyunsaturated fatty acids include, but are not limited to 4,7,10,13,16,19-docosahexaenoic acid (DHA; C22:6n-3; indicating 22 carbons, 6 double bonds, first double bond at position 3); 7,10,13,16,19 docosapentaenoic acid (C22:5n-3; DPA), 5,8,11,14,17-eicosapentaenoic acid (EPA; C20:5n-3); 8,11,14,17-eicosatetraenoic acid (ETA;C20:4n-3); 9,12,15 octadecatetraenoic acid (alpha linolenic acid, ALA; C18:3n-3), 6,9,12,15 octadecatetraenoic acid (stearidonic acid , SDA; 18:4n3). Compositions of the present invention may include highly enriched sources of such compounds, such as flax oil, Perilla oil (source of alpha linolenic acid), or the like. In such cases, it is preferable that such compositions contain less than about 50%, preferably less than about 25%, and more preferably less than about 10% of any omega-6 poly-unsaturated fatty acid that may be present in the mixture.

Omega-9 polyunsaturated fatty acids include, for example, 5,8,11-eicosatrienoic acid, an omega-9 fatty acid that has anti-inflammatory properties, and is produced in potentially commercial quantities by Suntory Ltd. (Osaka, JP). Other omega-fatty acids include 6,9 octadecadienoic acid and 8,11-eicosadienoic acid. U.S. Pat. No. 5,981,588, incorporated herein by reference, describes anti-allergic properties of these compounds and methods for obtaining such compounds.

A “stoichiometric amount” of a compound in a composition or formulation is used to mean an amount of such a compound that is greater than a trace amount, or more specifically, at least greater than about 0.025-0.05%, preferably greater than about 1%, still preferably greater than about 0.5%, more preferably greater than about 1-2% of the weight of active components in the composition or mixture. By way of example but not limitation, tocopherols are sometimes used as anti-oxidants for other compounds in a mixture. In such cases, the amount of the tocopherol present in the mixture may be on the order of 0.025-0.05% of the total mixture, and in such mixture, on the order of 0.06% of the active ingredient(s) in the mixtures.

As used herein amounts “effective to reduce premenstrual symptoms” or “effective amounts” is meant that the composition is or all components of a composition are present in a final concentration sufficient for reducing one or more premenstrual symptoms, such as, for example, edema, or a biomarker of PMS, such as CRP or WBC count. This amount includes, but is not limited to, a concentration that acts as a complete prophylaxis or treatment for one or more of the common premenstrual symptoms described herein. An effective amount can be administered in one or more administrations. For purposes of this invention, an effective amount of a composition or an effective amount of all components of a composition is an amount that is sufficient to ameliorate, stabilize, reverse, slow or delay premenstrual symptoms.

By “tocopherol” is meant any of a family of molecules which are characterized by a 6-chromanol ring structure and a side chain at the 2 position. A “beta-tocopherol enriched tocopherol composition”, as used herein refers to the beta-tocopherol as being enriched with respect to total tocopherols in the composition. Tocopherols possess a 4′,8′,12′-trimethyltridecyl phytol side chain. As used herein, the term “tocopherol” encompasses, but is not limited to:

alpha-tocopherol, [2R-2R*(4R*,8R*)]-3,4-dihydro-2,5,7,8-tetramethyl-2-(4,8,12-trimethyltridecyl)-2H-1-benzopyran-6-ol; 2,5,7,8-tetramethyl-2-(4′,8′,12′-trimethyltridecyl)-6-chromanol; 5,7,8-trimethyltocol, Fernholz (1937) J. Am. Chem. Soc. 59:1154 and 60:700;

beta-tocopherol, 3,4-dihydro-2,5,8-trimethyl-2-(4,8,12-trimethyltridecyl)-2H-1-benzopyran-6-ol; 2,5,8-trimethyl-2-(4,8,12-trimethyltridecyl)-6-chromanol; 5-8-dimethyltocol; cumotocopherol; neotocopherol; p-xylotocopherol;

gamma-tocopherol, 3,4-dihydro-2,7,8-trimethyl-2-(4,8,12-trimethyltridecyl)-2H-1-benzyopyran-6-ol; 2,7,8-trimethyl-2-(4,8,12-trimethyltridecyl)-6-chromanol; 7,8-dimethyltocol; o-xylotocopherol;

delta-tocopherol, [2R-[2R*(4R*,8R*)]]-3,4-dihydro-2,8-dimethyl-2-(4,8,12-trimethyltridecyl)-2H-1-benzo-pyran-6-ol; 8-methyltocol;

epsilon-tocopherol, [R-(E,E)]-3,4-dihydro-2,5,8-trimethyl-2-(4,8,12-trimethyl-3,7,11-tridecatrienyl)-2H-1-benzopyran-6-ol; 2,5,8-trimethyl-2-(4,8,12-trimethyltrideca-3,7,11-trienyl)chroman-6-ol; 5-methyltocol;

zeta1-tocopherol, 3,4-dihydro-2,5,7,8-tetramethyl-2-(4,8,12-trimethyl-3,7,11-tridecatrienyl)-2H-1-benzopyran-6-ol; 2,5,7,8-tetramethyl-2-(4,8,12-trimethyl-3,7,11-tridecatrienyl)-6-chromanol; 5,7,8-trimethyltocotrien-3′,7′,11′-ol;

zeta2-tocopherol, 3,4-dihydro-2,5,7-trimethyl-2-(4,8,12-trimethyltridecyl)-2H-1-benzopyran-6-ol; 2,5,7-trimethyl-2-(4,8,12-trimethyltridecyl-6-chromanol; 5,7-dimethyltocol; and

eta-tocopherol, 3,4-dihydro-2,7-dimethyl-2-(4,8,12-trimethyltridecyl)-2H-1-benzopyran-6-ol; 2,7-dimethyl-2-(4,8,12-trimethyltridecyl)-6-chromanol; 7-methyltocol. See The Merck Index (1996), Twelfth Edition, Merck & Co., Whitehouse Station, N.J., pp. 1620-1621 and 1712, and references cited therein. Other tocopherols include xi1-, xi2-, and sigma-tocopherols.

A “tocopherol” for use in the present invention can alternatively be a mixture of tocopherols. These mixtures include without limitation mixtures of stereoisomers of a single tocopherol (e.g., + and − stereoisomers of gamma-tocopherol (+/−) indicates a racemic mixture) or mixtures of structurally distinct tocopherols (e.g., delta- plus gamma-tocopherol).

By a “gamma-, beta-, or delta-tocopherol enriched tocopherol composition” is meant a composition that comprises at least 60%, at least 70%, at least 80%, at least 90%, or at least 95% gamma-, beta-, or delta-tocopherol, respectively. In some embodiments of the present invention, a tocopherol enriched tocopherol composition is one comprising less than 50% alpha-tocopherol, less than 45% alpha-tocopherol, less than 40% alpha-tocopherol, less than 35% alpha-tocopherol, less than 30% alpha-tocopherol, less than 25% alpha-tocopherol, less than 20% alpha-tocopherol, less than 15% alpha-tocopherol, less than 10% alpha-tocopherol or less than 2% alpha-tocopherol.

A “non-alpha tocopherol enriched tocopherol composition” is a composition that comprises at least 60%, at least 70%, at least 80%, at least 90%, or at least 95% of a tocopherol which is not alpha tocopherol, such as gamma-, beta-, or delta-tocopherol, respectively. In some embodiments of the present invention, a non-alpha tocopherol enriched tocopherol composition is one comprising less than 25% alpha-tocopherol, less than 20% alpha-tocopherol, less than 15% alpha-tocopherol, preferably less than 10% alpha-tocopherol or, more preferably, less than 55 or even 2% alpha-tocopherol.

By “treatment” or “treating” is meant any treatment of a disease or disorder, in a mammal, including: preventing or protecting against the disease or disorder, that is, causing, the clinical symptoms of the disease not to develop; inhibiting the disease, that is, arresting or suppressing the development of clinical symptoms; and/or relieving the disease, that is, causing the regression of clinical symptoms. A “treatment group” is a group that is being administered or has been administered a composition of the present invention or all components of a composition.

Mediacaments and Formulations

It is a discovery of the present invention that a combination of a tocopherol, particularly a non-alpha tocopherol, such as gamma-tocopherol, beta-tocopherol, and/or delta-tocopherol, and a highly unsaturated fatty acid, such as an omega-9 or an omega-3 polyunsaturated fatty acid, such as docosahexaenoic acid (DHA), is effective in reducing inflammatory symptoms associated with PMS, PMDD, endometriosis, post-partum depression, perimenopause, menopause, and administration of hormonal contraceptives. In particular, such formulations reduce CRP in women taking oral contraceptives. Other components of such formulations may include a mineral, particularly a divalent cation such as magnesium, and/or a flavonoid. Exemplary components of such formulations are described below.

Non-Alpha Tocopherols

Formulations or medicaments of the present invention may include a pure tocopherol or a non-alpha-tocopherol enriched tocopherol composition or mixture, namely a gamma-, delta- or beta-tocopherol, or a tocopherol derivative, or a mixture of tocopherols and/or tocotrienols that is enriched in a non-alpha tocopherol (i.e., where alpha-tocopherol comprises less than 25%, preferably less than 10% of tocopherols, and more preferably less than 2% of total tocopherols present in the medicament or other formulation of interest). Such compositions, when combined with one or more of the additional components of the formulation, are particularly efficacious in ameliorating certain symptoms of PMS as exemplified herein. In particular, non-alpha tocopherols that are particularly effective in anti-inflammatory compositions of the present invention include gamma, delta, and beta tocopherol. Other tocopherol derivatives, in accordance with the present invention, include known metabolites of tocopherols, for example, alpha- and gamma-tocopherol metabolites 2,5,7,8-tetramethyl-2-(2′-carboxyethyl)-6-hydroxychroman and 2,7,8-trimethyl-2-(2′-carboxyethyl)-6-hydroxychroman.

Other tocopherols useful in formulations of the invention may be determined empirically, with reference to the cellular anti-inflammatory assay described herein.

Tocopherols are chemical entities which, in general, contain a 6-chromanol ring structure and a side chain at the 2-position. Prototypical tocopherols include alpha-, beta-, delta- and gamma-tocopherol. The tocopherols have the general formula:

Tocopherols:

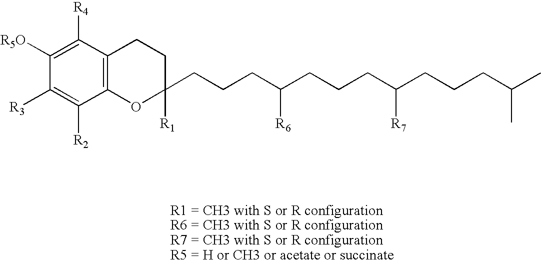

| | R2 | R3 | R4 |
|---|---|---|---|
| Alpha | CH3 | CH3 | CH3 |
| Gamma | CH3 | CH3 | H |
| Beta | CH3 | H | CH3 |
| Delta | CH3 | H | H |

Alpha-, gamma-, beta-, and delta-tocopherol have the structure as shown in Brigelius-Flohe, et al., 1999, The FASEB Journal, vol. 13: 1145.

As discussed herein, prototypical tocopherols include alpha-, beta-, gamma- and delta-tocopherol. In general, supplements that contain “Vitamin E” are generally understood to be composed predominantly of alpha-tocopherol. Tocopherols and their derivatives can vary by the number and position of alkyl groups, double bonds and other substituents and variations on the ring and side chain. In preferred embodiments, the tocopherol component of formulations of the present invention is predominantly a gamma-tocopherol, a beta-tocopherol, or a delta-tocopherol. In another preferred embodiment, the tocopherol component is made up of “mixed tocopherols,” such as those that are isolated from natural sources, with the proviso that such mixed tocopherol component will preferably contain or be supplemented to contain less than about 10%, preferably less than 5% alpha tocopherol, or more preferably less than 2% alpha tocopherol. Tocopherols may be obtained from a variety of sources, including Cargill, Incorporated (Minnetonka, Minn.), which processes a 95% pure gamma-tocopherol product, or Cognis Nutrition and Health (Cincinnati, Ohio), which markets a 92% pure gamma-tocopherol product.

Tocopherol derivatives may be constructed according to methods known in the chemical arts. In this context, an “alkyl” is a cyclic, branched or straight chain chemical group containing only carbon and hydrogen, such as methyl, butyl and octyl. Alkyl groups can be either unsubstituted or substituted with one or more substituents, e.g., halogen, alkoxy, acyloxy, amino, hydroxyl, mercapto, carboxy, or benzyl. Alkyl groups can be saturated or unsaturated at one or several positions. Typically alkyl groups will comprise 1 to 8 carbons, preferably 1 to 6, and more preferably 1 to 4 carbon atoms. Additional tocopherols can be constructed by conjugation to the ring structure or side chain of various other moieties, such as those containing oxygen, nitrogen, sulfur and/or phosphorus. Tocopherol derivatives can also be made, as known in the art, by modifying the length of the side chain from that found in prototypical tocopherols such as alpha-, beta-, delta- and gamma-tocopherol. Tocopherols can also vary in stereochemistry and saturation of bonds in the ring structure and side chain. Additional tocopherol derivatives, including prodrugs, can be made by conjugation of sugars or other moieties to the side chain or ring structure; these can serve any of a number of functions, including increasing solubility and increasing functional activity of the tocopherol. Thus, as is understood in the art, the invention encompasses the use of tocopherol derivatives in which substitutions, additions and other alterations have been made in the 6-chromanol ring and/or side chain, with the proviso that the derivatives maintain at least one functional activity of a tocopherol, such as antioxidant activity or ability to counteract sterility in animals. More preferably, by way of guidance, tocopherol derivatives useful in the invention will have CRP-lowering activity, such as in a cellular assay of CRP production, as described in Example 1, herein, either alone or in combination with an omega-3 fatty acid or an omega-6 fatty acid, as described further below.

An exemplary mixed tocopherol composition can be obtained, for example from Cargill Incorporated [Minnetonka, Minn.], and contains 62% gamma tocopherol, 28% delta tocopherol, 8% alpha tocopherol and less than 2% beta tocopherol. Additional mixed tocopherols from natural and transgenic sources are described, for example in PCT Publication WO 00/10380, incorporated herein by reference. Preferably, such mixed tocopherols will consist of less than 10%, preferably less than 5% alpha-tocopherol, and more preferably less than 2% alpha-tocopherol. Such mixed tocopherols may contain tocotrienols or other tocopherol-like derivatives in addition to the tocopherols mentioned above. Soybean oil is a particularly preferred natural source of mixed tocopherols of the invention; other preferred sources may include palm oil, corn oil, whole grain corn, safflower oil, rapeseed oil, whole wheat flour, or castor bean oil. Cargill and other commodities processors are sources for many of these materials. Preferred transgenic sources, as described in PCT Publication WO 00/10380, incorporated herein by reference, include soybean oil, oil palm oil, rapeseed oil, corn oil, and whole grain corn. Other natural and transgenic, enriched or otherwise artificially engineered sources will be readily apparent to the practitioner, with the guidance of the compositional guidance provided herein.

In further embodiments, the tocopherol component may be a metabolite of gamma-, delta- or beta-tocopherol, either in its administered or in vivo transformed form. One exemplary metabolite of gamma tocopherol is gamma-carboxy ethyl hydroxy chroman (gamma-CEHC), such as is further described by U.S. Pat. No. 6,083,982, incorporated herein by reference. The present invention also provides compositions comprising a gamma-tocopherol metabolite, a beta-tocopherol metabolite, and/or a delta-tocopherol metabolite, such as are well known in the art.

Derivatives of these compounds include, but are not limited to structural derivatives, as described above, as well as salts, including but not limited to succinate, nicotinate, allophanate, acetate, and phosphate salts of the tocopherols described herein. Salts also include pharmaceutically acceptable salts. Derivatives also include quinone derivatives and prodrug forms of tocopherols, such as those described in U.S. Pat. No. 5,114,957. Additional tocopherols and derivatives thereof are described in, e.g., U.S. Pat Nos. 5,606,080 and 5,235,073. Preparations of various tocopherols are described in, e.g., U.S. Pat. Nos. 5,504,220, 4,978,617, and 4,977,282. Various tocopherols are commercially available, for example from Sigma Chemical Co., St. Louis, Mo.

In the body of a subject, gamma-tocopherol breaks down into metabolites, including for example, the metabolites described in Wechter et al. U.S. Pat. Nos. 6,150,402; 6,083,982; 6,048,891; and 6,242,479, specifically incorporated herein in their entireties. In particular, the present invention encompasses the use of gamma-tocopherol enriched tocopherol compositions that further comprise a gamma-tocopherol metabolite such as gamma-CEHC, racemic gamma-CEHC and (S) gamma-CEHC.

In the body of a subject, beta-tocopherol breaks down into metabolites. In particular, the present invention encompasses the use of compositions that comprise a beta-tocopherol metabolite such as 2,5,8-trimethyl-2-(2-carboxyethyl)-6-hydroxychroman (beta-CEHC). The present invention encompasses the use of compositions that comprise a beta-tocopherol metabolite such as beta-CEHC, racemic beta-CEHC and (S) beta-CEHC.

In the body of a subject, delta-tocopherol breaks down into metabolites. In particular, the present invention encompasses the use of compositions that comprise a delta-tocopherol metabolite such as delta-CEHC, racemic delta-CEHC and (S) delta-CEHC.

Poly-Unsaturated Fatty Acid Component

Exemplary highly unsaturated fatty acids that may be used in the formulations and methods of the invention include omega-3 fatty acids, such as all-cis 4,7,10,13,16,19-docosahexaenoic acid (DHA; C22:6n-3); 5,8,11,14,17-eicosapentaenoic acid (EPA; C20:5n-3); or 5,8,11,14,-eicosatetraenoic acid. Other exemplary omega-3 fatty acids are described herein. Alternatively, the highly unsaturated fatty acid may be an omega-9 fatty acid such as 5,8,11-eicosatrienoic acid (C20:3n-9, also known as “Mead acid”).

Polyunsaturated fatty acids are commercially available from a number of vendors. DHA can be obtained, for example, from Martek Biosciences Corporation (Columbia, Md.). Martek provides a microalgae-derived product, a 40% DHA product marketed as “NEUROMINS.” U.S. Pat. Nos. 5,492,938 and 5,407,957, incorporated herein by reference, describe methods of producing DHA from microalgae. DHA from other sources, including cold-water ocean fish, sea mammals, and range-fed poultry, as well as other omega-3 fatty acids, are also commercially available from sources known in the art. Generally such sources of DHA provide a mixture of omega-3 fatty acids, sometimes with other components. While various sources may be used, in accordance with the present invention, it may be preferred that formulations containing DHA be prepared or obtained from a source, such as microalgae (Martek) to provide a relatively high ratio of DHA:EPA, preferably at least about 10:1. Similarly, medicaments comprising less than 10% of omega-6-fatty acids, such as linolenic acid or linoleic acid, may also be preferred, according to another aspect of the invention.

Omega-9 polyunsaturated fatty acids have been characterized as anti-allergy compounds in U.S. Pat. No. 5,981,588, incorporated herein by reference, and are available from Suntory Ltd. (Osaka, Japan). These compounds may be components of a salutary medicament, according to a further aspect of the present invention.

Other highly unsaturated fatty acids are known in the art, for example U.S. Pat. No. 6,376,688, incorporated herein by reference, describes certain anti-malarial, neutrophil stimulatory polyunsaturated fatty acids characterized by their enhanced stability in vivo, by virtue of exhibiting slower metabolic turnover, for example, 8-hydroperoxy-5Z,9E,11Z,14Z-eicosatetraenoic acid.

Derivatives of the aforementioned polyunsaturated fatty acids are also suitable for use in the invention, for example, esters of DHA, glycerides of DHA, and the like, such as described in U.S. Pat. No. 5,436,269, incorporated herein by reference.

Flavonoid Component

In another embodiment, the formulation or medicament may include at least one flavonoid, such as is defined in the “Definitions” section herein. In some embodiments, the compositions comprise at least two such flavonoids. In yet other preferred embodiments, the flavonoids include chrysin, diosmin, hesperetin, luteolin, rutin, or quercetin. In additional embodiments, the flavonoids are hesperetin and quercetin, singly, or more preferably, in combination. Thus, in some embodiments of the present invention, compositions comprise gamma-tocopherol, hesperetin, quercetin and DHA. Ranges and approximate dosages are described below.

Flavonoids comprise a class of polyphenolic substances based on a flavan nucleus, generally comprising 15 carbon atoms, arranged in three rings as C₆-C₃-C₆. There are a number of chemical variations of the flavonoids, such as, the state of oxidation of the bond between the C2-C3 position and the degree of hydroxylation, methoxylation or glycosylation (or other substituent moieties) in the A, B and C rings and the presence or absence of a carbonyl at position 4. Flavonoids include, but are not limited to, members of the following subclasses: chalcone, dihydrochalcone, flavanone, flavonol, dihydroflavonol, flavone, flavanol, isoflavone, neoflavone, aurone, anthocyanidin, proanthocyanidin (flavan-3,4-diol) and isoflavane.

Flavanones contain an asymmetric carbon atom at the 2-position and flavanones include, but are not limited to, narigenin, naringin, eriodictyol, hesperetin and hesperidin. Dihydroflavonols include, but are not limited to, taxifolin (dihydroquercetin). Flavones include, but are not limited to, chrysin, diosmin, luetolin, apigenin, tangeritin and nobiletin. Flavonols include, but are not limited to, kampferol, quercetin and rutin. Flavanes include, but are not limited to, catechin and epi-gallocatechin-gallate. Isoflavones include, but are not limited to, biochanin, daidzein, glycitein and genistein.

In some embodiments, compositions comprise a flavanone. In further embodiments, compositions comprise the flavanone hesperetin.

In other embodiments, compositions comprise flavonols, such as, quercetin.

In yet further embodiments, the compositions comprise an isoflavone. In other embodiments, the compositions comprise a flavone. In further embodiments, the compositions comprise a flavonol.

Hesperetin and hesperidin are flavonoids found in citrus, such as lemons, grapefruits, tangerines and oranges, and may be extracted from the peel of citrus or synthesized according to the process described by Shinoda, Kawagoye, C.A. 23:2957 (1929); Zemplen, Bognar, Ber., 75,1043 (1943) and Seka, Prosche, Monatsh., 69, 284 (1936). Hesperetin may also be prepared by the hydrolysis of hesperidin (see, for example, U.S. Pat. No. 4,150,038).

Daidzein is a flavonoid isolated from red clover (Wong (1962) J. Sci. Food Agr. 13:304) and from the mold Micromonospora halophytica (Ganguly et al. Chem. & Ind. (London) 197, 201. Additional descriptions of isolation of daidzein from various plant products can be found in Hosny et al. (1999) J. Nat. Prod. 62: 853-858 and Walz (1931) Ann. 489:118. Synthesis of daidzein is described in Farkas et al. (1959) Ber. 92:819. Daidzein is an inactive analog of the tyrosine kinase inhibitor genistein (Sargeant et al. (1993) J. Biol. Chem. 268:18151). Daidzein is also a phytoestrogen, recently suggested to play a role in preventing special types of cancer. See, for example, Sathyamoorthy et al. (1994) Cancer Res. 54:957; Zhou et al. (1999) J. Nutr. 129: 1628-1635 and Coward et al. (1993) J. Agric. Food Chem. 41:1961. Daidzein also has anti-estrogen properties (Anderson et al. (1998) Baillieres Clin. Endocrinol. Metab. 12: 543-557). Daidzein also acts as an anti-oxidant, inhibiting lipid peroxidation. Arora et al. (1998) Arch. Biochem. Biophys. 356: 133-41; and Hodgson et al. (1999) Atherosclerosis 145: 167-72.

Biochanin A can be isolated from red clover (Pope et al. (1953) Chem. & Ind. (London) 1092 and Wong (1962) J. Sci. Food. Agr. 13:304) and its structure is described by Bose et al. (1950) J. Sci. Ind. Res. 9B:25. Biochanin A has some anticancer properties. Lyn-Cook et al. (1999) Cancer Lett. 142: 111-119; Hammons et al. (1999) Nutr. Cancer 33: 46-52; Yin et al. (1999) Thyroid 9: 369-376. Biochanin A also has anti-oxidant properties, including the ability to inhibit lipid peroxidation. Toda et al. (1999) Phytother. Res. 13: 163-165.

Flavonoids isolated and purified from natural sources or chemically synthesized may be used in the invention. Methods to isolate and identify flavonoids have been described, for example, in Markham et al. (1998) pp.1-33, in Flavonoids in Health and Disease, Rice-Evans and Packer, eds. Marcel Dekker, Inc. Many flavonoids are commercially available from sources such as Funakoshi Co., Ltd. (Tokyo), Sigma Chemical Co. (St. Louis, Mo.) and Aldrich Chemical Co. (Milwaukee, Wis.). Generally, hesperetin, hesperidin, quercetin, diosmin, daidzein, chyrsin, luteolin, biochanin and rutin are available from commercial sources.

Also suitable in the present invention are derivatives of flavonoids. For example, derivatives of a flavonoid differ from the flavonoid in structure. These differences can be, as non-limiting examples, by addition, substitution or re-arrangement of hydroxyl, alkyl or other group. As a non-limiting example, a flavonoid derivative can have additional alkyl groups attached. In addition, flavonoid derivatives include compounds which have been conjugated to another chemical moiety, such as a sugar or other carbohydrate. Other suitable moieties contain oxygen, nitrogen, sulfur, and/or phosphorus. Derivatives of flavonoids can be produced, for example, to improve its solubility, reduce its odor or taste, or to ensure that the compound is free of toxicity. A flavonoid can also be conjugated to another moiety to form a prodrug. In a prodrug, a flavonoid is conjugated to a chemical moiety which, for example, aids in delivery of the flavonoid to the site of activity (e.g., a particular tissue within the body). This chemical moiety can be optionally cleaved off (e.g., enzymatically) at that site.

Hesperetin derivatives are described in, for example, Esaki et al. (1994) Biosci. Biotechnol. Biochem. 58:1479-1485; Scambia et al. (1990) Anticancer Drugs 1:45-48; Bjeldanes et al. (1977) Science 197:577-578; Honohan et al. (1976) J. Agric. Food Chem. 24:906-911; and Brown et al. (1978) J. Agric. Food Chem. 26:1418-1422.

While differing from the flavonoid in structure, derivatives of the flavonoid will retain at least one activity of the flavonoid. For hesperetin and hesperetin derivatives these activities include anti-oxidant and anti-free radical activity (Saija et al. (1995) Free Radic. Biol. Med. 19:481-486). Activities associated with hesperetin include, but are not limited to, the following. Hesperetin is an antilipolytic in rat adipocytes (Kuppusamy et al. (1993) Planta Med. 59:508-512) and has activity in controlling sebum production and in treatment of side disorders (U.S. Pat. No. 5,587,176). Hesperetin may act in inhibiting mammary tumorigenesis and proliferation of breast cancer cells (Guthrie et al. (1998) Adv. Exp. Med. Biol. 439:227-236; So et al. (1997) Cancer Lett. 112:127-133). Hesperetin inhibits 7-(ethoxycoumarin)-deethylase activity in rat liver microsomes (Moon et al. (1998) Xenobiotica 28:117-126) and also reduces the susceptibility of membrane Ca²⁺-ATPase to thyroid hormone stimulation. Hesperetin increases ocular blood flow (Liu et al. (1996) J. Ocul. Pharm. Ther. 12:95-101). Hesperetin inhibits myeloperoxidase ('T Hart et al. (1990) Chem. Biol. Interact. 73:323-335) and inhibits 3-hydroxy-3-methylglutaryl CoA reductase (U.S. Pat. No. 5,763,414). Hesperetin derivatives retain at least one of these activities.

Derivatives of diosmin include diosmin heptakis (hydrogensulfate) aluminum complex, and diosmin octakis (hydrogen sulfate) aluminum complex, as described in U.S. Pat. Nos. 5,296,469; and 4,894,449. Another derivative of diosmin is its aglycone form, diosmetin, 5,7-dihydroxy-2-(3-hydroxy-4-methoxypenyl)-4H-1-benzopyran-4-one. See The Merck Index (1989), Eleventh Edition, p. 520, and references cited therein. Derivatives of diosmin also include salts thereof. A synthetic diosmin derivative, LEW-10, is described in Azize et al. (1992) Chem. Phys. Lipids 63:169-77.

While differing from diosmin in structure, diosmin derivatives will retain at least one activity of diosmin. Diosmin is commonly administered to protect blood vessels and prevent and/or treat herpesvirus attacks. Diosmin also has free radical scavenger activity (Dumon et al. Ann. Biol. Clin. 52: 265-270, 1994); is an antilipoperoxidant (Feneix-Clerc et al. Ann. Biol. Clin. 52:171-177, 1994); inhibits 5′-nucleotidase (Kavutcu et al. Pharmazie 54:457-459, 1999); attenuates lipopolysaccharide cytotoxicity in cell culture (Meizig et al. Pharmazie 54:29809, 1999); probably affects cytochrome P450 activity (Teel et al. Cancer Lett. 133:135-141, 1998 and Ciolino et al. Cancer Res. 58:2754-2760, 1998). The combination of diosmin and hesperidin, known as DAFLON™ 500, has been alleged to exhibit anti-inflammatory, anti-free radical, venotonic and vasculoprotective activities, in addition to attenuating reperfusion injury. Guillot et al. Pancreas 17:301-308, 1998; Amiel et al. Ann. cardiol. Angeiol. 47:185-188, 1998; Nolte et al. Int. J. Microcirc. Clin. Exp. 17 (suppl. 1): 6-10, 1997; Delbarre et al. Int. J. Microcirc. Clin. Exp. 15 (suppl. 1): 27-33, 1995; Bouskela et al. Int. J. Microcirc. Clin. Exp. 15 (suppl. 1):22-6, 1995; and Friesenecker et al. Int. J. Microcirc. Clin. Exp. 15 (suppl. 1):17-21, 1995. The combination of diosmin and hesperidin is also allegedly useful for treating hemorrhoids. U.S. Pat. No. 5,858,371. A diosmin derivative retains at least one of these activities.

Derivatives of daidzein, biochanin A and other compounds described herein include compounds which are chemically and/or structurally similar, but non-identical to such compounds, and which share at least one function of those compounds. Numerous derivatives of daidzein are known in the art. These include daidzein 7-glucoside, or daidzin; and the aglucon of daidzein. Glycosylated and methoxylated derivatives of daidzein are described in Arora et al. (1998). Chlorinated derivatives of daidzein are described in Boersma et al. Arch. Biochem. Biophys. 368: 265-275, 1999. Additional derivatives are described in Lapcik et al. Steroids 62: 315-320, 1997; Joannou et al. J. Steroid. Biochem. Mol. Biol. 54: 167-184, 1995; Keung Alcohol Clin. Exp. Res. 17: 1254-1260, 1993; Smit et al. J. Biol. Chem. 267: 310-318, 1992; Shao et al. Yao Hsueh Hsueh Pao 15: 538-547, 1980 and King et al. Am. J. Clin. Nutr. 68: 1496S-1499S, 1998. Numerous derivatives of biochanin A are also described in the art, in, for example, chlorinated derivatives described in Boersma et al. (1999).

Mineral Component

Compositions of the present invention may also include a mineral supplement, such as magnesium. Other mineral supplements may be used, for example copper, zinc, selenium, molybdenum, manganese, chromium, iodine, iron and combinations thereof. In formulations of the present invention, divalent ions, such as calcium and magnesium, zinc, and manganese are preferred; however, there is some indication that calcium may compete for or otherwise inhibit magnesium functionality in this regard (See Abraham, cited above).

In an exemplary embodiment of the present invention, compositions comprise gamma-tocopherol, DHA and magnesium; other compositions contain gamma-tocopherol, hesperetin, quercetin, DHA and magnesium. Ranges and approximate dosages are described below.

Excipients and Preparations

In further embodiments, formulations and medicaments of the present invention may comprise an excipient suitable for use in dietary or nutritional supplements. For example, in studies carried out in support of the present invention (Example 4), formulations were prepared in high oleic sunflower oil (A. C. Humko (TRISUN 80; Cordova, Tenn.)). Other acceptable nutritional excipients are well known in the art, and may include, without limitation, binders, coatings, disintegrants, and hydrocolloids, which may be used advantageously to provide desired properties. There are many competitive vendors of such products; one major supplier is FMC Corporation (Philadelphia, Pa.). Formulations may also comprise an excipient suitable for pharmaceutical uses; such excipients are well known in the art (See, e.g., Remington's Pharmaceutical Sciences).

Medicaments of the present invention may be conveniently packaged in a capsule, tablet, or pill, for oral ingestion, in accordance with one preferred aspect of the invention, according to methods well known in the art. By way of example, but not limitation, such oral forms may include be prepared as solid dosage forms, sustained and controlled release forms, liquids, or semi-solids. Optionally, medicaments, especially multi-component medicaments as described herein, may be packaged in a plurality of capsules or tablets for oral ingestion.

In another preferred embodiment, formulations of the invention may be incorporated into a daily “vitamin” regimen. For example, the components can incorporated into standard multi-vitamins, or may be included as additional capsules in a multi-vitamin supplement package which includes a variety of dietary supplements or “pills” in a pre-wrapped format, such as in a sealed cellophane packet containing pre-defined dosage(s). Alternatively, the various components of the formulation can be separately bottled and sold, or suggested to be purchased, in combination.

Along the same lines, for certain uses, such as ameliorating inflammatory symptoms of hormonal contraceptive use, medicaments of the present invention may be packaged with, and/or co-administered with oral contraceptives.

The compositions, as described above, can be prepared as a medicinal preparation (such as an aqueous solution for injection) or in various other media, such as foods for humans or animals, including medical foods and dietary supplements. A “medical food” is a product that is intended for the specific dietary management of a disease or condition for which distinctive nutritional requirements exist. By way of example, but not limitation, medical foods may include vitamin and mineral formulations fed through a feeding tube to cancer or burn victims (referred to as enteral administration or gavage administration). A “dietary supplement” refers to a product that is intended to supplement the human diet and is typically provided in the form of a pill, capsule, tablet or like formulation. By way of example, but not limitation, a dietary supplement may include one or more of the following ingredients: vitamins, minerals, herbs, botanicals, amino acids, dietary substances intended to supplement the diet by increasing total dietary intake, and concentrates, metabolites, constituents, extracts or combinations of any of the foregoing. Dietary supplements may also be incorporated into food stuffs, such as functional foods designed to promote tissue health or to prevent inflammation. If administered as a medicinal preparation, the composition can be administered, either as a prophylaxis or treatment, to a patient in any of a number of methods. The subject compositions may be administered alone or in combination with other pharmaceutical agents and can be combined with a physiologically acceptable carrier thereof. The effective amount and method of administration of the particular formulation can vary based on the individual subject, the stage of disease, and other factors evident to one skilled in the art. During the course of the treatment, the concentration of the subject compositions may be monitored to insure that the desired level is maintained.

Generally, the route(s) of administration useful in a particular application are apparent to one of skill in the art. Routes of administration include, but are not limited to, oral, topical, dermal, transdermal, transmucosal, epidermal, parenteral, and gastrointestinal.

For administration, the invention includes subject compositions suitable for oral administration including, but not limited to, nutritionally accepted vehicles, such as soft gel caps, pharmaceutically acceptable tablets, capsules, powders, solutions, dispersions, or liquids. In preparing the compositions in oral dosage form, any of the usual media may be employed. For oral liquid preparations (e.g., suspensions, elixirs, and solutions), media containing, for example, water, oils, alcohols, flavoring agents, preservatives, coloring agents and the like may be used. Carriers such as starches, sugars, diluents, granulating agents, lubricants, binders, disintegrating agents, and the like may be used to prepare oral solids (e.g., powders, capsules, pills, tablets, and lozenges). Controlled release forms may also be used. Because of their ease in administration, tablets, pills, and capsules represent the most advantageous oral dosage unit form, in which case solid carriers are obviously employed. If desired, tablets may be sugar coated or enteric coated by standard techniques.

For rectal administration, the subject compositions may be provided as suppositories, as solutions for enemas, or other convenient application. Suppositories may have a suitable base comprising, for example, cocoa butter or a salicylate.

Formulation for vaginal administration may be presented as pessaries, tampons, creams, gels, pastes, foams or spray formulations containing in addition to the active ingredient such carriers as are known in the art to be appropriate.

Otherwise, the subject compositions may be administered intravascularly, arterially or venous, subcutaneously, intraperitoneally, intraorganally, intramuscularly, by dermal patch, or the like.

For administration, the formulations may conveniently be presented in unit dosage form and may be prepared by any methods well known in the art of pharmacy. Such methods include the step of bringing into association the active ingredients with the carrier that constitutes one or more accessory ingredients. In general, the formulations are prepared by uniformly and intimately bringing into association the active ingredients with liquid carriers or finely divided solid carriers or both, and then if necessary shaping the product.

For oral administration, suitable subject compositions include, but not limited to, pharmaceutically acceptable tablets, capsules, powders, solutions, dispersions, or liquids. Also, the subject compositions may be compounded with other physiologically acceptable materials which can be ingested including, but not limited to, foods, including, but not limited to, food bars, beverages, powders, cereals, cooked foods, food additives and candies.

When the composition is incorporated into various media such as foods, it may simply be orally ingested. The food can be a dietary supplement (such as a snack or wellness dietary supplement) or, especially for animals, comprise the nutritional bulk (e.g., when incorporated into the primary animal feed).

The amount of the composition ingested, consumed or otherwise administered will depend on the desired final concentration. Typically, the amount of a single administration of the composition of the invention can be about 0.1 to about 1000 mg per kg body weight, or about 0.5 to about 10,000 mg per day. Any of these doses can be further subdivided into separate administrations, and multiple dosages can be given to any individual patient. A typical dosage for vitamin E (alpha tocopherol) administration is 100-1000 mg/day for an adult human. However, various different dosages are described in scientific publications; see, for example, Ng et al. Food Chem. Toxicol. 37: 503-8, 1999; Ko et al. Arch. Phys. Med. Rehabil. 80: 964-7, 1999; Chen et al. Prostaglandins Other Lipid Mediat. 57: 99-111, 1999; and Thabrew et al. Ann. Clin. Biochem. 36: 216-20, 1999.

To determine the optimum concentration for any application, conventional techniques may be employed. Thus, for in vitro and ex vivo use, a variety of concentrations may be used and various assays employed to determine the degree of inflammation.

Formulations of the present invention adapted for oral administration as medicaments may be presented as discrete units such as capsules, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredients or components may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropylmethylcellulose), lubricant, inert diluent, preservative, disintegrant (e.g. sodium starch glycollate, cross-linked povidone, cross-linked sodium carboxymethylcellulose) surface-active or dispersing agent. Molded tablets may be made by moulding in a suitable machine a mixture of the powdered compound moistened with an inert liquid diluent. The tablets may optionally be coated or scored and may be formulated so as to provide controlled release of the active ingredients therein using, for example, hydroxypropylmethylcellulose in varying proportions to provide the desired release profile.

The subject compositions may be administered parenterally including intravascularly, arterially or venous, subcutaneously, intradermally, intraperitoneally, intraorganally, intramuscularly, or the like.

Formulations for parenteral administration include aqueous and non-aqueous isotonic sterile injection solutions which may contain buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Another type of formulation is an emulsion. Emulsifiers may be nonionic, anionic or cationic and examples of emulsifiers are described in, for example, U.S. Pat. Nos. 3,755,560, and 4,421,769.

Liposomal formulations are also useful for the compositions of the present invention. Such compositions can be prepared by combining gamma-tocopherol, and/or metabolite thereof, and/or derivative thereof, and/or mixtures thereof, with a phospholipid, such as dipalmitoylphosphatidyl choline, cholesterol and water according to known methods, for example, as described in Mezei et al. (1982) J. Pharm. Pharmacol. 34:473-474, or a modification thereof. Epidermal lipids of suitable composition for forming liposomes may be substituted for the phospholipid. To determine the optimum concentration for any particular application or method of administration, conventional techniques may be employed.

The above-mentioned compositions and methods of administration are meant to describe but not limit the methods and compositions of the present invention. The methods of producing various compositions and devices are within the ability of one skilled in the art and are not described in detail here.

Ranges of Components in Formulations of the Invention

Generally, amounts of tocopherols administered in a dietary supplement form will be within a range of doses that would be found in the diets of humans. Higher amounts may be used in regimens that are administered or overseen by clinical professionals. While multi-component dietary supplements generally provide about 100-200% of the Dietary Reference Intake for vitamin E, which is currently set at 15 mg/day, higher dosages of tocopherols may be administered, under appropriate regulatory and toxicological guidelines.

Formulations of the present invention may include a non-alpha tocopherol, as defined above, such as gamma tocopherol, in the range of 10 milligrams (mg) to 10,000 mg, more generally in the range of 20 mg to 1000 mg. Preferably, dosages of between about 100 mg and 500 mg will be ingested daily. Dosages of other non-alpha tocopherols may be determined empirically, with reference to gamma tocopherol. For example, in studies carried out in support of the present invention (Example 4), subjects self-administered 300 mg of a gamma-tocopherol enriched tocopherol mixture daily, in conjunction with other components of the formulation of the present invention. Other tocopherols may be substituted in such a regimen, and overall efficacy compared to that of gamma-tocopherol in relieving such premenstrual symptoms as were measured in the PMS study described herein. More generally, it is anticipated that tocopherols that are preferred for use in the present invention will exhibit CRP-lowering activity in vitro, for example, activity comparable to that of gamma-tocopherol in a CRP lowering assay, such as the cell assay detailed in Example 1A herein.

By way of example, according to the present invention, the tocopherol component of an effective formulation may include 300 mg of “mixed tocopherols” available as a commodity, for example, as a combination of 200 mg of gamma-tocopherol, and the remainder a mixture of delta and/or beta tocopherol, with less than about 10%, preferably less than 5%, and more preferably less than 2% alpha-tocopherol alpha-tocopherol present in the mixture.

According to a further aspect of the invention, an omega-3 polyunsaturated fatty acid, such as docosahexaenoic acid (DHA), or an omega-9 polyunsaturated fatty acid, such as 5,8,11-eicosatrienoic acid (Mead acid), is added to the tocopherol to produce an effective medicament for ameliorating inflammatory symptoms associated with PMS, PMDD, perimenopause, menopause, and the like. This component can be incorporated with the tocopherol(s) in a single administration, or can be given separately, in a regimen designed to provide relief from such symptoms, such as premenstrual symptoms.

American average dietary intake of DHA (10-60 mg/day) is low compared to intake in countries where fish or fish products comprise higher percentages of the diet. Toxicological studies have demonstrated that 50× these levels (e.g., 3.6 gm DHA per day) can be ingested by humans with no apparent toxicities (Grimsgaard S, et al. Am J Clin Nutr 66:649-659, 1997). Generally, ranges of about 10-10,000 mg, or more specifically, about 50-2000 mg, or 100-1000 mg will be preferred. In studies carried out in support of the present invention, women ingested approximately 800 mg DHA daily, or just over 10× an average American dietary amount. Appropriate dosages of other polyunsaturated fatty acids can be estimated with reference to this study, based on known safe ingestion levels, or may be determined empirically, with the guidance provided herein.

Flavonoids may be added to formulations of the present invention, either in combination or in separate administered doses, as described herein. There is a wide variety of flavonoids present in foods commonly ingested by humans. Particularly rich sources of flavonoids include onions, apples, tea and cabbage. While there are no DRI or UL (upper limit) values established for flavonoids, American dietary intakes are estimated at below 20 mg/day. In studies carried out in support of the present invention, women suffering from premenstrual symptoms ingested a combination of flavonoids amounting to 100 mg total supplemental flavonoids, specifically quercetin and hesperetin. Other flavonoids can be substituted in this regimen, as described above. More generally, flavonoids will be added in the range of 10-1000 mg, 20-800 mg, 50-500 mg, 50-300 mg, 100-200 mg, less than 1000 mg, less than 800 mg, less than 500 mg, less than 300 mg, less than 200 mg, greater than 10 mg, greater than 20 mg, greater than 30 mg, greater than 50 mg, greater than 100 mg.

A mineral, preferably a divalent ion such as magnesium, may be added to the tocopherol and polyunsaturated fatty acid components mentioned above. Magnesium dietary intake is generally in the range of 50-500 mg/day. Leafy green vegetables and whole grains are particularly robust dietary sources of magnesium. The United States adult DRI for magnesium is 400 mg/day; however, most adults (especially women) ingest far less. The 100 mg in the formulation is 25% of the DRI. Accordingly, formulations of the invention may include magnesium in the range of 10-1000 mg, 20-800 mg, 50-400 mg, 50-300 mg, 100-200 mg, less than 1000 mg, less than 800 mg, less than 400 mg, less than 250 mg, less than 200 mg, greater than 10 mg, greater than 20 mg, greater than 30 mg, greater than 50 mg, greater than 100 mg. Other minerals can be substituted with reference to their DRIs and Upper Limits (Reference: Food and Nutrition Board, Institute of Medicine, Washington, D.C.), since toxicity may occur at very high doses of certain minerals.

Kits

In further embodiments, formulations of the present invention may be incorporated into kits. Such kits will include the components of medicaments of the invention, as defined herein, particularly where the components of the formulation are present in a plurality of tablet or capsule forms packaged in separate containers. Alternatively, individual dosage small packets, each containing the appropriate dose of each of the multiple components of the desired formulation may be provided. Such kits may further include instructions for determining levels of WBC and/or CRP, so that a care provider, or the subject, can monitor her levels of these, or other inflammatory biomarkers of interest.

In a further modification, the kit may include measurement means for determining WBC or CRP. Exemplary means are described herein (e.g., Example 1A) or are readily available in the art. Conveniently, such means may include ELISA or EIA-based detection methods.

Methods of Treatment

The present invention further includes methods of treating subjects suffering from inflammatory symptoms associated with PMS, PMDD, perimenopause, menopause, or the like, and ameliorating or reducing at least one premenstrual symptom selected from the group: dysmenorrhea, acne, retention of body fluids (bloating), breast tenderness, dizziness, fatigue, headache, hot flashes, nausea, diarrhea, constipation, heart palpitations, swelling of the hands and feet, and cramps. Affective and cognitive symptoms can be present in the form of mood swings, angry outbursts, violent tendencies, anxiety, nervousness, tension, difficulty concentrating, depression, crying easily, depression, food cravings, forgetfulness, irritability, increased appetite, mood swings, and increased emotional sensitivity. Studies carried out in support of the present invention indicate that women who, in the course of a clinical study, self-administered medicaments of the present invention over a 3 month period reported significantly lower incidence of the various symptoms mentioned above (Example 4;FIG. 1).

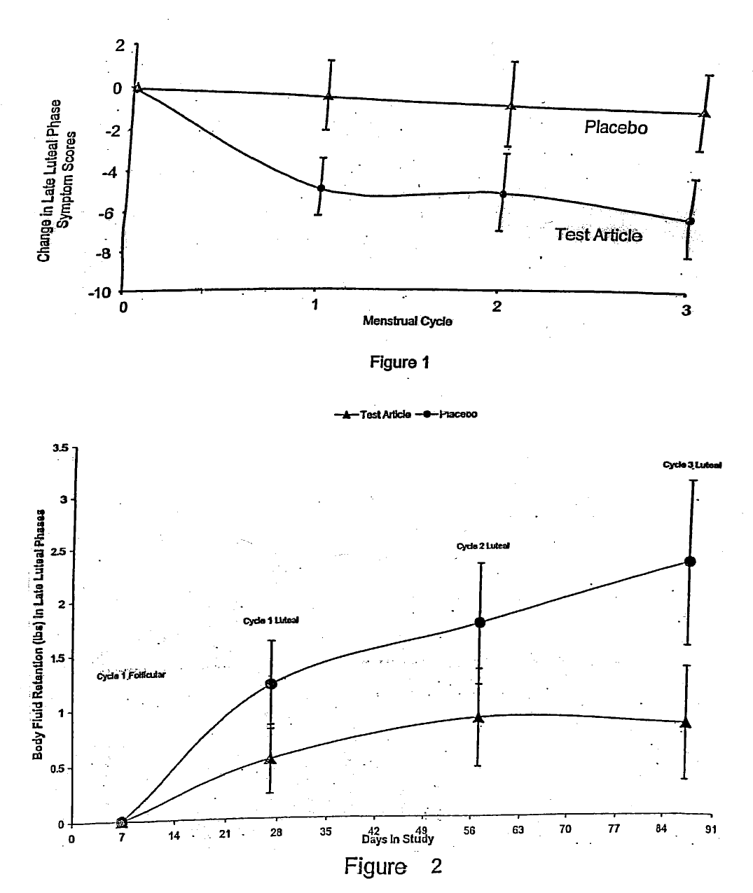

Preferably, the method of the invention will affect bloating, edema and/or weight gain associated with the luteal phase of a woman's cycle. In studies carried out in support of the present invention (Example 4), it was observed that women taking test article, as opposed to placebo controls, experienced significantly less edema and premenstrual weight gain during luteal phase (c.f., FIG. 2).

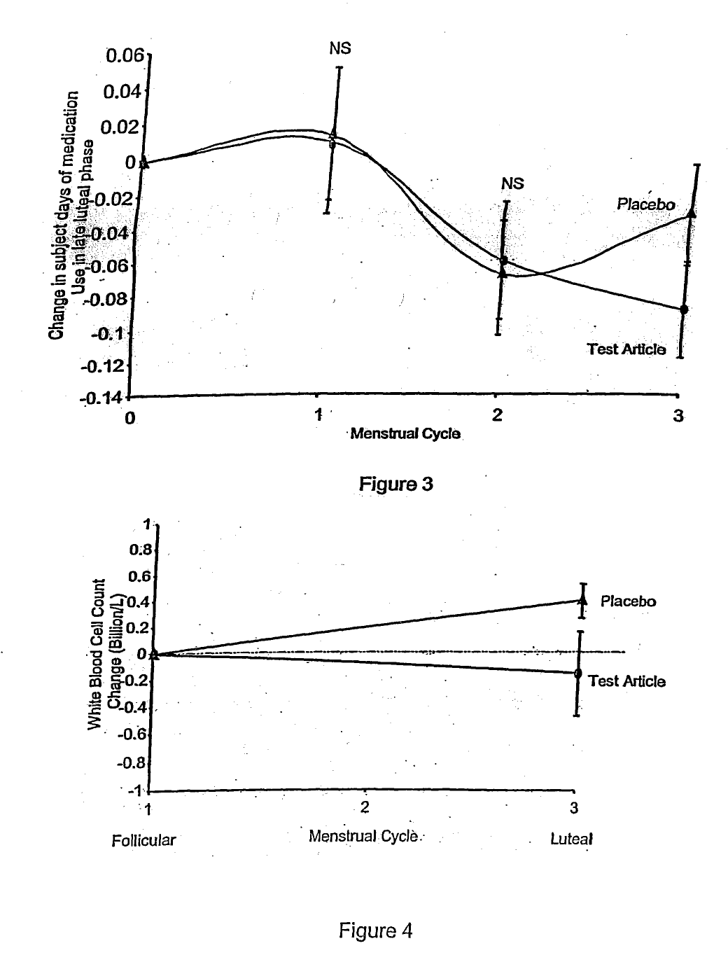

Another advantage of the method of the invention, illustrated in the data shown as FIG. 3, is that women taking the formulation decreased their ad libidum consumption of non-steroidal analgesic and/or anti-inflammatory agents, such as aspirin and ibuprofen.

Further, a perimenopausal and menopausal female reported experiencing reduced symptoms associated with these conditions, including acne, retention of body fluids (bloating), fatigue, headache, hot flashes, and certain affective and cognitive symptoms (mood swings, angry outbursts, anxiety, tension, depression, crying easily, irritability and emotional sensitivity), when she self-administered a medicament formulation according to the present invention (Example 6) over a period spanning approximately 12 months.

Generally, the method of the present invention includes administering to a female subject in need of such treatment, a formulation as described in the previous section. Minimally, the formulation will comprise a tocopherol, preferably a non-alpha tocopherol, in combination with a polyunsaturated fatty acid, preferably an omega-3 polyunsaturated fatty acid. The formulation may also include a mineral, such as magnesium and/or a flavonoid, such as discussed above.

In some embodiments, compositions are administered in one dosing of a single formulation and in other embodiments, compositions are administered in multiple dosing of a single formulation. In some embodiments, all components of a composition are administered together in a single formulation, that is, all components are present in a single formulation and in other embodiments, all components of a composition are administered separately in two formulations or multiple formulations, such that all components are administered to a subject within a specified time period. In some embodiments, the time period is between about 3 hours to about 6 hours. In other embodiments, the time period is between about 6 hours and 12 hours. In additional embodiments, the time period is between about 12 hours and 24 hours. In yet further embodiments, the time period is between about 24 hours and 48 hours. The administration of separate formulations can be simultaneous or staged throughout a specified time period, such that all ingredients are administered within the specified time period.

For example, for administration of the following components: 300 mg of mixed tocopherols (180 mg gamma-tocopherol; 30 mg alpha-tocopherol; and 90 mg delta-tocopherol); 33 mg hesperetin; 66 mg quercetin; and 800 mg docosahexaenoate (DHA) and 100 mg magnesium per day per mammalian subject, the ingredients are administered as a) one composition comprising all components in a single dosing; b) one composition containing less than the total of all components in two or multiple dosings within a specified time period, such as for example two dosings per day per mammalian subject of formulations comprising 150 mg of mixed tocopherols (90 mg gamma-tocopherol; 15 mg alpha-tocopherol; and 45 mg delta-tocopherol); 17 mg hesperetin; 33 mg quercetin; and 400 mg docosahexaenoate (DHA); c) two or multiple compositions administered in one dose per day per mammalian subject, such as for example, 300 mg of mixed tocopherols (180 mg gamma-tocopherol; 30 mg alpha-tocopherol; and 90 mg delta-tocopherol) administered in one composition once a day along with 300 mg of flavonoids (100 mg hesperetin; 200 mg quercetin) administered in one composition once a day along with 800 mg DHA administered in one composition once per day; d) two or multiple compositions administered in a staged manner throughout the day, such as for example, 300 mg of mixed tocopherols (180 mg gamma-tocopherol; 30 mg alpha-tocopherol; and 90 mg delta-tocopherol) administered in one composition once a day along with 300 mg of flavonoids (100 mg hesperetin; 200 mg quercetin) administered in one composition once per day along with a composition comprising 200 mg DHA administered 4 times staged throughout the day; or e) each component in its own composition administered either once a day if the composition comprises the total desired amount of the component to be administered per day or multiple times a day if the composition comprises less than the total desired amount of ingredient to be administered per day with administrations throughout the day up to the total amount of components to be administered.

Illustrative examples of ranges of components in formulations and methods of the invention include:

gamma-tocopherol or a gamma-tocopherol enriched tocopherol composition or beta-tocopherol or a beta-tocopherol enriched composition or delta-tocopherol or a delta-tocopherol enriched composition or a gamma-, beta-, or delta-tocopherol metabolite, ranging from in the lower limit at least about 10 mg, at least about 50 mg, at least about 100 mg, at least about 150 mg, at least about 200 mg, at least about 250 mg, at least about 300 mg, at least about 350 mg, or at least about 400 mg per mammalian subject per day and ranging from in the upper limit not greater than about 2000 mg, not greater than about 1500 mg, not greater than about 1250 mg, not greater than about 1000 mg, not greater than about 750 mg, not greater than about 500 mg per mammalian subject per day, wherein the lower limit and the upper limit are selected independently and in some embodiments the range of gamma-tocopherol or a gamma-tocopherol enriched tocopherol composition or beta-tocopherol or a beta-tocopherol enriched composition or delta-tocopherol or a delta-tocopherol enriched composition or a gamma-, beta-, or delta-tocopherol metabolite is from about 10 to about 1000 mg, or from about 50 to about 600 mg, or from about 100 to about 400 mg per mammalian subject per day; and

DHA ranging from in the lower limit at least about 25 mg, at least about 50 mg, at least about 75 mg, at least about 100 mg, at least about, 125 mg, at least about 150 mg, at least about 175 mg, at least about 200 mg, at least about 250 mg, at least about 275 mg, at least about 300 mg, at least about 325 mg, at least about 350 mg, or at least about 400 mg per mammalian subject per day and ranging from in the upper limit not greater than about 1500 mg, not greater than about 1250 mg, not greater than about 1000 mg, not greater than about 900 mg, and not greater than about 800 mg per mammalian subject per day wherein the lower limit and the upper limit are selected independently and in some embodiments, the range of DHA is from about 100 to about 1000 mg, or about 200 to about 900 mg, or about 400 to about 800 DHA mg per mammalian subject per day.

A formulation or method of the invention may also include:

A mineral, ranging from a lower limit of the DRI of such mineral to an upper limit of about 10× the DRI. More specifically, the mineral may be magnesium, for which a DRI of 400 mg has been established. In this case, ranges from above may be used.

In addition, the formulation of method of the invention may include:

A flavonoid, such as hesperetin or quercetin, ranging from in the lower limit, at least about 10 mg, at least about 15 mg, at least about 25 mg, at least about 50 mg, at least about 75 mg, at least about 100 mg at least about 125 mg, at least about 150 mg, at least about 200 mg, or at least about 250 mg per mammalian subject per day and ranging from in the upper limit not greater than about 1000 mg, not greater than about 750 mg, not greater than about 500 mg, not greater than about 475 mg, not greater than about 450 mg, not greater than about 425 mg, not greater than about 400 mg, not greater than about 375 mg, not greater than about 350 mg, not greater than about 325 mg, or not greater than about 300 mg wherein the lower limit and the upper limit are selected independently and in some embodiments the range of hesperetin or quercetin is from about 10 to about 500 mg, or from about 25 to about 200 mg, or from about 50 to about 100 mg per mammalian subject per day.

The formulation or method of the invention may also include an oral contraceptive.

The below are illustrative compositions encompassed within the present invention given as total mgs per day administered to a mammalian subject. In the below examples, the components may be administered together in one composition or administered separately in two or multiple compositions simultaneously or staged throughout the day.

Composition I

300 mg of mixed tocopherols (180 mg gamma-tocopherol; 30 mg alpha-tocopherol; and 90 mg delta-tocopherol); 800 mg DHA, 33 mg hesperetin; 67 mg quercetin; 100 mg magnesium.

Composition II

300 mg of mixed tocopherols (180 mg gamma-tocopherol; 30 mg alpha-tocopherol; and 90 mg delta-tocopherol); 800 mg docosahexaenoate (DHA); and

100 mg magnesium.

Composition III

300 mg of a gamma-tocopherol enriched composition (greater than 270 mg gamma-tocopherol); 800 mg DHA 100 mg hesperetin and 200 mg quercetin.

Composition IV

300 mg of a gamma-tocopherol enriched composition (greater than 270 mg gamma-tocopherol); 800 mg DHA.

The foregoing compositions are only exemplary and should not be construed to limit the invention. Activity of a composition of the present invention, or activity of components administered in methods of the present invention, can be experimentally tested, for example, in an assay which measures the ability of the composition to reduce CRP or circulating white blood cell levels in vitro or to reduce WBC count in vivo in mid-luteal phase female subjects. Assays which measure the ability of a test composition to ameliorate premenstrual symptoms in vivo are detailed in Examples.

Specific Biomarkers and Assays for Inflammation

A number of proximal mediators of the inflammatory response have been identified and include the inflammatory cytokines, interleukin-1-alpha (IL-1α) (U.S. Pat. No. 6,210,877) and tumor necrosis factor alpha (TNF-alpha), as described in U.S. Pat. Nos. 5,993,811 6,210,877 and 6,203,997. Other molecules have been reported for use as markers of systemic inflammation, including for example, C-reactive protein (CRP; Ridker et al. N. E. J. M. 342(12):836-43, 2000; Spanheimer supra); certain cellular adhesion molecules such as sICAM-1 (U.S. Pat. No. 6,049,147); and B61 (U.S. Pat. No. 5,688,656). Other markers associated with inflammation include leukotriene, thromboxane, isoprostane, and soluble TNF-30 receptors. A further aspect of the present invention is the observation that certain of these inflammatory markers are elevated during mid-luteal phase, and that reduction of such markers can serve as an objective biomarker for reduction of premenstrual symptoms. Thus, according to yet a further aspect, the invention includes a method for assessing efficacy of therapies and formulations designed to ameliorate premenstrual symptoms.

There exist various commercial sources that produce reagents for assays for C-reactive protein, for example, but not limited to, CalBiochem (San Diego, Calif.). B61 is secreted by endothelial cells, fibroblasts and keratinocytes in response to lipopolysaccharide and the pro-inflammatory cytokines IL-1 and TNF. The B61 gene product is not, however, induced in response to other agents such as growth factors and interferon, thus induction of B61 is thus highly specific to inflammation (U.S. Pat. No. 5,688,656). The presence of B61 transcript can be detected directly by in situ hybridization using probes of encoding cDNA. Alternatively, the B61 protein can be measured in biological fluids such as plasma, cerebrospinal fluid or urine using an antibody-based assay. These assay procedures known in the art and described in particular in U.S. Pat. No. 5,688,656 are useful in both prognostic and diagnostic applications.

A novel biomarker of lipid peroxidation is the recently described class of compounds called isoprostanes, products of the non-enzymatic interaction of reactive oxygen species with the polyunsaturated fatty acid arachidonate (Morrow et al., Biochem. Pharmacol. 51:1-9, 1996). A common isoprostane, 8-iso-PGF₂α, has been demonstrated to have potent bioactivity in promoting inflammation, platelet activation, and vasospasm. Under physiological conditions, isoprostanes are produced in total quantities that exceed the structurally related prostaglandins, and they exert their bioactivity both through prostaglandin receptors and via isoprostane-specific receptors (Kunapuli, 1998). Single phytonutrients such as α-tocopherol (Davi et al, Circulation 99:224-229, 1999) and fish oil (Mori et al, Metabolism, 48:1402-8, 1999), used in other inflammatory conditions (eg, diabetes), have demonstrated modest anti-isoprostane effects.

With this perspective, isoprostanes may serve as appropriate primary endpoints for an intervention study directed against reactive oxygen species as mediators of premenstrual symptoms, particularly symptoms attributable to endometriosis (Van Langendonckt, A., et al., Fertil. Steril. 77(5):861-870, 2002). Due to their participation in inflammatory and tissue injury pathways, other secondary endpoints include inflammatory markers C-reactive protein (CRP), white blood cells (WBC), and interleukin-6 (IL-6); tissue injury markers such as creatine kinase (CK) and lactate dehydrogenase (LDH); and subjective measures of muscle soreness. Other markers may include arachidonic acid, particularly as measured in the membranes of various cells readily sampled in a subject, such as red blood cell membranes, white blood cell membranes or mucous cell membranes (buccal cells, nasal cells, rectal cells, vaginal cells).

According to this embodiment of the invention, such biomarkers may be readily measured according to methods well characterized in the art. An effective premenstrual composition or formulation is one that lowers one or more of the various markers mentioned above.

The physiology of antioxidant protection in animals is clearly multi-layered. Some antioxidants are closely associated with membrane lipids or lipoproteins, while others are distributed into the cytosol. Some are enzymatically regenerated while others are expended. And, not all anti-oxidants have anti-isoprostane activity. Indeed, supplemental vitamin C and N-acetyl-cysteine have been shown to increase markers of oxidative stress in humans after an inflammatory condition (Childs et al., Free Radical Biology & Medicine 31:745-53, 2001).

In studies carried out in support of the present invention, in a randomized, double-blind, placebo-controlled study, a formulation consisting of 300 mg of mixed tocopherols (180 mg gamma-tocopherol; 30 mg alpha-tocopherol; and 90 mg delta-tocopherol); 33 mg hesperetin; 66 mg quercetin; 800 mg docosahexaenoate (DHA); and 100 mg magnesium was given to healthy human female subjects with regular menstrual cycles and diagnosed as suffering from moderate to severe PMS standard diagnostic criteria for at least 6 months prior to the study. Example 4 provides details of the study, including the various biomarker parameters monitored. The examples demonstrate that mid-luteal WBC count was reduced in formulation-treated subjects, as compared to subjects who received placebo. In addition, there was a reduction in CRP levels in treated, as compared to placebo control subjects.

U.S. Pat. No. 6,040,147 describes both prognostic and diagnostic applications of the measurement of levels of particular molecules including certain cytokines (e.g. interleukins 1-17) and cellular adhesion molecules (e.g. sICAM, integrins, ICAM-1, ICAM-3, BL-CAM, LFA-2, VCAM-1, NCAM and PECAM). The presence of such markers may be determined by methods well known in the art, including ELISA (enzyme linked immunosorbent assay) and other immunoassays and can be measured in body fluid, for example, blood, lymph, saliva and urine. U.S. Pat. No. 6,180,643 also describes the use of molecules such as IL-1, TNF-α as markers.

Methods of Using Formulations of the Invention

The compositions of the present invention are administered to a mammalian subject to maintain and promote healthy and/or normal levels of proteins or biomarkers, such as, for example, CRP, WBC, leptin, certain cytokines associated with inflammation as described herein, TNF-alpha and B61 that are associated with inflammation in a subject. Healthy or normal ranges of such proteins are known in the art. See for example, U.S. Pat. No. 6,040,147 which provides healthy or normal ranges for CRP. See Anim-Nyame N, et al., (Hum Reprod; 15:2329-32, 2000) for a description of leptin elevation in PMS, and methods for measuring same.

For example, compositions of the present invention are administered to a mammalian subject at risk for developing elevated levels of CRP, such as individuals taking oral contraceptives, in order to maintain healthy or normal levels of CRP. The formulations of the present invention are administered to a mammalian subject to reduce elevated levels of proteins or biomarkers associated with mid-luteal phase premenstrual symptoms, for example, WBC, CRP, and/or certain cytokines associated with inflammation as described herein.

Further, formulations of the present invention are administered to a subject to reduce certain premenstrual symptoms, such as edema and bloating, and premenstrual weight gain. In particular, formulations of the present invention are effective in reducing the following specific premenstrual symptoms during late luteal phase: behavioral symptoms (angry outbursts, arguments, violent tendencies, anxiety, tension, nervousness, confusion, difficulty concentrating, crying easily, depression, mood swings, overly sensitive behavior); fatigue. Women who received formulations of the invention also self-administered reduced amounts of non-steroidal anti-inflammatory drugs (NSAIDs, e.g., aspirin, ibuprofen and the like) as compared to their placebo-treated counterparts (FIG. 3). Treated subjects also reported significantly less edema (bloating) during premenstrual period than placebo-control treated subjects (FIG. 2).

The compositions of the present invention are administered to a subject in amounts to reduce premenstrual symptoms mid-late luteal phase, such as edema, fatigue, and the behavioral symptoms mentioned above. The subject may be a female who has historically suffered from PMS, PMDD or who is at risk for developing premenstrual symptoms, such as, for example, women in the terminal 15, 10, or preferably 2-8 or more preferably 4-8 years of menses (peri-menopausal subjects). The methods encompass administering a composition of the present invention to a subject. The amount administered and the duration of the treatment are effective to minimize the physical and/or behavioral premenstrual symptoms, such as symptoms reported by subjective assessment, or, alternatively or in addition, as measured by for example, CRP levels, WBC, IL-6 levels, TNF-alpha levels, or isoprostane levels, as described herein. For example, the formulation may be taken or administered daily throughout the menstrual cycle, as illustrated in the studies described herein; alternatively the formulation may be taken or administered just prior to onset of symptoms, such as at the beginning of the luteal phase. The formulation may also be taken or administered after onset of symptoms. Preferably, subjects will begin medication prior to onset of symptoms, however, to enhance overall wellbeing and avoid onset of severe symptoms. Thus, it is anticipated that as a result of such treatment the incidence and/or severity of premenstrual symptoms is minimized.

Similarly compositions of the present invention may be administered to women with elevated levels of CRP, due to their intake of oral contraceptives.

The following examples are provided to illustrate, but not limit, the invention.

### EXAMPLES

### Example 1

### Cellular Inflammation

This example provides exemplary assays for measuring inflammatory reaction in a cell line. Specifically, this assay provides a predictive measure of bioactivity for formulations and/or components of formulations for use in the compositions and formulations of the present invention.

A. Human Hep3B Cells—CRP Assay

Hep3B Cell Line was obtained from the American Type Culture Collection (ATCC Catalog No. HB-8064). The Hep3B cell line was derived from liver tissue of an 8-year-old African-American male. The cells are epithelial in morphology and produce tumors in nude mice. The cells produce alpha-fetoprotein, hepatitis B surface antigen, albumin, alpha-2-macroglobulin, alpha-1-antitrypsin, transferrin, plasminogen, complement C3 and alpha-lipoprotein (Knowles B B, et al., Science, 209:497-499, 1980). This cell line has been widely used to study hepatocyte cytokine and acute phase protein release (e.g., Damtew B, et al, J Immunol. 150:4001-4007, 1993).

HEP3B cells are grown in Minimum Essential Medium (MEM; GIBCO) supplemented with 10% Fetal Bovine Serum (FBS; Hyclone), 1× Penicillin/Streptomycin (GIBCO, Cat #.15140-122) and 0.1 mM non-essential amino acids (GIBCO, Catalog No. 11140-050). Prior to culture, cells are thawed and transferred to warm medium according to standard methods known in the art.

HEP3B cells were incubated in flasks at 37° C. with 5% CO₂ in an air atmosphere incubator. HEP3B growth media was changed every 2 days until the cells reach 70-80% confluence (approx. 3-4 days). For assay, the cells were transferred to 96-well plates, seeded at 5000 cells per well in culture media, and left to grow for 7 days in a 37° C. incubator (air supplemented with 5% CO₂). Media was replaced daily until assay.

Test compounds were diluted into “Stimulus Buffer” (MEM medium containing 0.1 mM non-essential amino acids, 1× penicillin/streptomycin, 10% FBS with 10 ng/ml IL-1β, 20 ng/ml IL-6 and 1 μM dexamethasone. Media was removed from the cells and was replaced with 200 μl of test dilution. Cells were returned to the incubator for three days at 37° C. CRP ELISA was then performed on supernatant from the cells, as described below.

Costar EIA/RIA plates were coated with rabbit anti-human CRP (DAKO) diluted 1:4000 in carbonate buffer (100 μl/well) for 45 minutes at 37° C. Plates were then washed 5× with CRP washing buffer (50 mM Tris-HCl, 0.3M NaCl, 0.5 Ml Tween-20, pH 8.0) using an automatic plate washer. In some cases, plates were dried, covered and refrigerated until use. Supernatant (100 μl) was removed from each well of the test plates and added to the corresponding well of a precoated ELISA plate.

100 microliters (μL) HRP-conjugated rabbit anti-human CRP (DAKO) diluted 1:500 (in CRP wash buffer) were added to each well, followed by incubation for 30 minutes at 37° C. Plates were washed 5× with CRP washing buffer using an automatic plate washer. 200 μL of 3,3′,5,5′-Tetramethyl Benzidine (TMB) liquid Substrate System (Sigma, St. Louis, Mo.) was added to each well, followed by incubation in the dark for 15 minutes at room temperature. Finally, 50 μL of 1M H₂SO₄ was added to each well and absorbance at 450 nm was immediately measured in a microtiter spectrophotometer.

CRP measured as above was normalized to cell count per well, using a cell viability assay, such as the Cell Tracker Green assay (Molecular Probes, Eugene, Oreg.). To do this, the remainder of the medium was removed from the cell test plates, cells were washed with 200 μl of pre-warmed 1× Hanks Basic Salt Solution (HBSS; GIBCO), and 100 μL of 5 μM Cell Tracker Green (Molecular Probes, Eugene, Oreg.) was added to each well. Plates were then incubated at 37° C. for 30 minutes. Cells were then washed twice with prewarmed 1× HBSS. Plates were immediately read using a Fluoroskan® flourometer with a 485 excitation/538 emission filter pair.

B. Cell-ELAM Assay

Endothelial-Leukocyte Adhesion Molecule (ELAM), also known as E-selectin, is expressed on the surface of endothelial cells. In this assay, lipopolysaccharide (LPS) and IL-1β are used to stimulate the expression of ELAM; test agents are tested for their abilities to reduce this expression, in accordance with studies showing that reduction of leukocyte adhesion to endothelial cell surface is associated with decreased cellular damage (e.g., Takada, M., et al., Transplantation 64: 1520-25, 1997; Steinberg, J. B., et al., J. Heart Lung Trans. 13:306-313, 1994).

Endothelial cells may be selected from any of a number of sources and cultured according to methods known in the art; including, for example, coronary artery endothelial cells, human brain microvascular endothelial cells (HBMEC; Hess, D. C., et al., Neurosci. Lett. 213(1): 37-40, 1996), or lung endothelial cells. Cells are conveniently cultured in 96-well plates. Cells are stimulated by adding a solution to each well containing 10 μg/ml LPS and 100 pg/ml IL-1β for 6 hours in the presence of test agent (specific concentrations and time may be adjusted depending on the cell type). Treatment buffer is removed and replaced with pre-warmed Fixing Solution® (100 μl/well) for 25 minutes at room temperature. Cells are then washed 3×, then incubated with Blocking Buffer (PBS+2% FBS) for 25 minutes at room temperature. Blocking Buffer containing Monoclonal E-Selectin Antibody (1:750, Sigma Catalog #S-9555) is added to each well. Plates are sealed and stored at 4° overnight. Plates are washed 4× with 160 μL Blocking Buffer per well. Second Antibody-HRP diluted 1:5000 in Blocking Buffer is then added (100 μL/well), and plates are incubated at room temperature (protected from light) for two hours. Plates are then washed 4× with Blocking Buffer before addition of 100 μL of ABTS Substrate solution at room temperature (Zymed, Catalog #00-2024). Wells are allowed to develop for 35 minutes, before measurement at 402 nm in a Fluoroskan® Reader with shake program for 10 seconds. Positive results are recorded as a decrease in ELAM concentration in tested wells, as compared to control wells.

C. Selection of Components

Formulation components selected from tocopherols, tocopherol derivatives, polyunsaturated fatty acids, minerals and flavonoids, as described herein, were tested in one or more of the assays described in Example 1. Compounds are selected for use in a formulation or treatment method of the invention, if they exhibit a potency in such assays that is equivalent to, or at least 1/10 as potent as the potency of the following components: gamma tocopherol, quercetin or hesperetin. This testing also provides basis for selecting relative dosages of each of the selected components. Such dosages can be selected with reference to the dosages provided for the standard components described herein, with further reference to known pharmacokinetic principles (See, e.g., Hardman & Limbird, Eds., Goodman & Gilman's The Pharmacological Basis of Therapeutics, 9^{th} Ed., McGraw-Hill, New York).

### Example 2

### Preparation of Soft Gelatin Capsules

Soft gelatin encapsulation of mixed tocopherols and DHA were carried out by a commercial manufacturer (Tishcon Corp., Westbury, N.Y.) using standard manufacturing practices known in the art under GMP guidelines. Briefly, raw materials were obtained from commercial sources (DHA, Martek Biosciences Corp., Columbia, Md.; Mixed Tocopherols and High Oleic Sunflower Oil, Cargill Incorporated, Minneapolis, Minn.). Weighed raw materials were placed into a mixer for blending according to standard methods known in the art. The mixed blend was milled and homogenized through colloidal mill according to manufacturing instructions. The liquid blend was discharged into a stainless steel tank

Shell Material:

Weight raw materials were charged to gelatin melter. Gelatin mass was prepared by stirring the mix blend for 2 to 2½ hours at 180° F. to 190° F. and under the proper vacuum. After gelatin mass was ready, it was discharged into the appropriate stainless steel tank and kept at 140 to 142° F. Viscosity of gelatin mass was then measured and recorded.

Encapsulation:

Encapsulation was processed according to encapsulation machine instructions and product specifications. During encapsulation, softgels were checked every 30 minutes for proper shell and fill weights, ribbon and seal thickness. Softgels from the encapsulation line were collected in trays and kept in a controlled drying room for 48 hours at 70-72 degrees F. and 25-30% relative humidity. After the drying process, capsules were visually inspected, then packed in boxes lined with plastic bags. A calculation of actual yield of capsules. and the percentage variation from theoretical was carried out as further quality assurance.

Mixed tocopherols (Cargill, Minnetonka, Minn.) comprising 62% gamma tocopherol, 28% delta tocopherol, 8% alpha tocopherol, and less than 2% beta tocopherol (by weight) were incorporated into softgel carriers. DHA was incorporated into separate softgel carriers. The standard capsule contained used in studies carried out in support of the present invention contained 300 mg mixed tocopherols or 200 mg DHA, with appropriate fillers. Study participants were asked to ingest 5 softgel capsules daily (1 tocopherol mix; 4 DHA softgels). Matching placebo softgel capsules were manufactured with high oleic sunflower oil (Cargill) incorporated in the place of mixed tocopherol and DHA, for use in control subjects. Compliance was monitored by measurement of DHA in the red blood cells of subjects.

### Example 3

### Preparation of Hard Capsules

For experiments carried out in support of the present invention, hard gelatin capsules were prepared using standard methods known in the art. The flavonoids quercetin and hesperetin were incorporated (33 mg and 66 mg, respectively) along with 167 mg magnesium oxide, with rice powder as filler for a total 400 mg capsule. For use in control studies carried out in support of the present invention, rice powder filler was used without further augmentation.

### Example 4

### Effects of Anti-Inflammatory Composition on PMS Symptoms

A clinical study was conducted, using healthy volunteers, to correlate inflammatory markers with a subjective assessment of PMS symptoms, and to determine the effect of administering formulations of the present invention on certain symptoms, specifically acne, bloating, breast tenderness, dizziness, fatigue, headache, hot flashes, nausea, diarrhea, constipation, heart palpitations, swelling of the hands and feet, and cramps. Affective and cognitive symptoms can be present in the form of mood swings, angry outbursts, violent tendencies, anxiety, nervousness, tension, difficulty concentrating, depression, crying easily, depression, food cravings, forgetfulness, irritability, increased appetite, mood swings, and increased emotional sensitivity. In accordance with a further embodiment of the present invention, surrogate markers of inflammation were quantitated in the subjects. Total WBC with differential count, red blood cell arachidonate and CRP were determined for each subject.

Patients with PMS received daily dosing of the test article (300 mg mixed tocopherol; 800 mg DHA; 33 mg hesperetin; 67 mg quercetin;100 mg magnesium) or placebo control for three consecutive menstrual cycles. Capsules for oral administration were taken daily. Compliance was validated by monitoring the DHA content in red blood cell membranes. A daily internet questionnaire recorded changes in symptom scores for the following symptoms:

acne

bloatedness

breast tenderness

dizziness

fatigue

headache

hot flashes

nausea, diarrhea, constipation

palpitations

swellings (hands, ankles, breast)

angry outbursts, violent tendencies

anxiety, tension nervousness

difficulty concentrating

crying easily

depression

food cravings (sweets, salts)

forgetfulness

irritability

increased appetite

mood swings

overly sensitive

wish to be alone

cramps (low abdominal/backache/general aches and pains)

The intervention trial used a randomized, placebo-controlled, double-blind parallel group design in which subjects were given a formulation or placebo during three consecutive menstrual cycles. Subjects were assessed at time points during the follicular and luteal phases of their menstrual cycles. The women were monitored during three menstrual cycles, and the effect of treatment or placebo on inflammatory markers and on the premenstrual symptoms noted above was recorded.

Subjects included in the study were healthy, non-smoking women with regular menstrual cycles and normal blood pressure. All subjects met the ICD 10 [define] criteria for moderate to severe PMS for at least 6 months prior to the study, as evidenced by physician medical history. Diagnosis was confirmed by prospective daily recording of menstrual-related symptoms for 3 cycles. Also by prospective daily menstrual diaries for 13 cycles, 70% of women met the DSM IV criteria for PMDD.

Subjects were randomly assigned to one of two treatment arms; with one group receiving placebo (500 ml gel caps containing high oleic sunflower oil and 400 mg hard-shell capsules containing rice flour) and the other receiving the test article (mixed Tocopherol, DHA, Hesperetin, Quercetin and Magnesium). Subjects recorded scores from 0 to 66 on a daily questionnaire to provide an assessment of their symptom status.

FIG. 1 shows the results of a study in which women selected as described above ingested daily capsules containing placebo ingredients (described above) or the following test article formulation components:

300 mg mixed tocopherol (65% gamma tocopherol, 25% delta, 10% alpha)

800 mg DHA

33 mg hesperetin

67 mg quercetin

100 mg magnesium oxide

Subjects who received active formulations of the present invention showed statistically significant improvement in overall symptomatology, as depicted in the graph of FIG. 1.

### Example 5

### Oral Contraceptive Usage and C-Reactive Protein

CRP levels were measured using stored samples from 30 healthy, premenopausal women who had previously participated in a randomized, crossover study of the effects of soy intake on sex hormone metabolism in women using OCs and non-users. The study protocol was approved by the Institutional Review Board: Human Subjects Committee of the University of Minnesota, and informed consent was obtained from all subjects prior to the start of the study. In summary, the participants (women aged 18-40 years from the university community) consumed their habitual diet or a soy-enriched diet for 2 menstrual cycles each. Soy consumption had no effect on sex hormone metabolism in OC or non-OC users (Martini et al., Nutrition and Cancer 34(2), 133-139, 1999). Non-OC users were trained in basal body temperature charting and ovulation testing for verification of follicular and luteal phases. Serum progesterone concentrations were used to confirm ovulation. Four fasting blood samples and 24-hr urine (2 mid-follicular and 2 mid-luteal) were collected from each participant over two menstrual cycles and were stored at −70° C. until laboratory analysis. OC users provided fasting blood samples on days 8 and 22 after menses. Plasma samples were later thawed and assayed for CRP by use of a high-sensitivity assay with a coefficient of variation <7.6% (Roberts et al.,Clinical Chemistry 46(4), 461-468, 2000).

Participants

For the present analysis, 30 of the 36 available women (20 OC users and 16 nonusers) were included because they had complete blood data. Women who used OC (n=18) reported that they had used OC for more than three months, with 75% reporting using OC for at least one year. Nine of the OC users were on three different triphasic combination pills (Triphasil-28, Ortho-Novum 7-7-7, or Ortho Tri-Cyclen 28). The other 9 participants were on 9 different formulations of single-dose pills; the drug preparations contained low-dose estrogen (0.020-0.035 mg ethinyl estradiol equivalents) combined with low-dose progestins (0.1-0.5 mg of dl-norgestrel equivalents). Formulations of OCs were combined due to small numbers of women reporting use of specific types. Non-OC users (n=12) reported regular menstrual cycles and no menstrual disorders for the last year, with cycle length ranging from 25 to 30 days, and not using OC for ≧6 months.

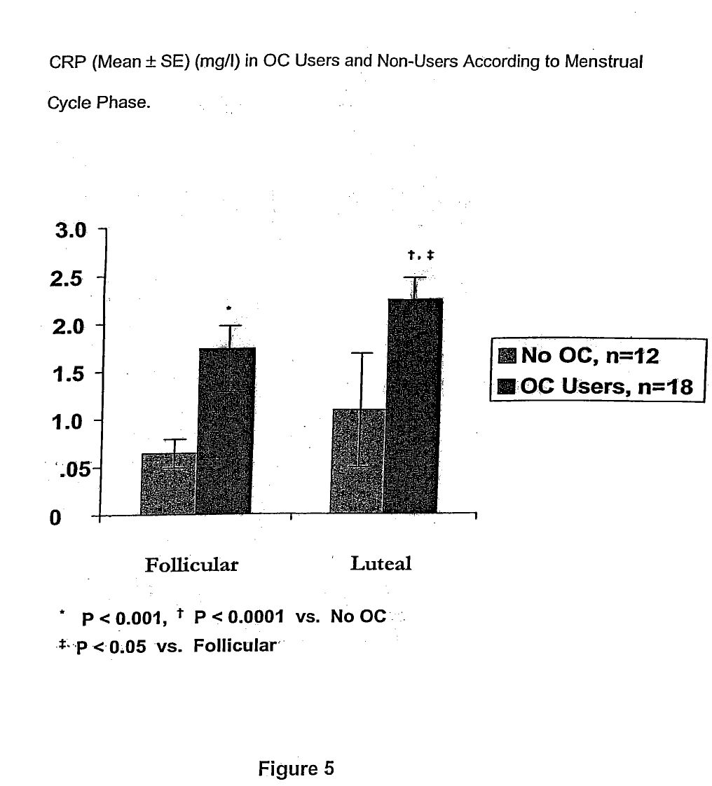

Statistical analyses were performed using statview (sas institute, inc. Cary, N.C.). Baseline characteristics were compared between participants according to oc use by a non-paired t-test. Primary analyses focused on the cross-sectional association between OC use and plasma CRP. Users and nonusers were compared on each diet assignment and during each menstrual cycle phase. Plasma CRP results were normalized by log transformation then analyzed for differences between OC users and non-OC users by three-way analysis of variance (anova) controlling for diet assignment (soy or control) and menstrual cycle phase (follicular or luteal). T-test was used for within group analyses of differences in CRP levels between soy and control diets and between luteal and follicular phases. Multiple regression was used to evaluate the relationship between OC use and CRP. For presentation, means and standard errors were transformed back to their original units. For all analyses, results were considered statistically significant at p <0.05. Results of these comparisons are shown in FIG. 5.

### Example 6

### Reduction of Peri-Menopausal and Menopausal Symptoms

A 49-year old female experiencing perimenopausal and menopausal symptoms self-administered a dosage of 400 mg of gamma-tocopherol-enriched tocopherol formulation (1 gelcap containing 300 of mixed tocopherols: 180 mg gamma-tocopherol; 30 mg alpha-tocopherol; and 90 mg delta-tocopherol ) and 800 mg of DHA (4 gelcaps containing 200 mg each), each morning. She noted a decrease in acne, retention of body fluids (bloating), fatigue, headache, hot flashes, and certain affective and cognitive symptoms (mood swings, angry outbursts, anxiety, tension, depression, crying easily, irritability and emotional sensitivity).

It is claimed:

1. A medicament for ameliorating or reducing inflammatory symptoms related to premenstrual syndrome (PMS), premenstrual dysphoric disorder (PMDD), perimenopause, menopause, or administration of hormonal contraceptives in a female mammalian subject, comprising a stoichiometric amount of a non-alpha tocopherol or tocopherol metabolite composition and an omega-3 poly-unsaturated fatty acid, wherein said tocopherol or tocopherol derivative composition and said omega-3 poly-unsaturated fatty acid are present in an amount effective to reduce an inflammatory biomarker in said subject, wherein said non-alpha tocopherol composition comprises no more than about 10% alpha tocopherol.

2. The medicament of claim 1, wherein said tocopherol composition comprises no more than about 5% alpha tocopherol.

3. The medicament of claim 1, wherein said tocopherol composition comprises no more than about 2% alpha tocopherol.

4. The medicament of claim 1, wherein said tocopherol composition is selected from the group consisting of a beta-tocopherol enriched tocopherol composition, a delta-tocopherol enriched tocopherol composition and a gamma-tocopherol enriched tocopherol composition.

5. The medicament of claim 1, wherein said tocopherol comprises a gamma-tocopherol-enriched tocopherol composition.

6. The medicament of claim 5, wherein said tocopherol composition comprises at least about 60% gamma-tocopherol.

7. The medicament of claim 5, wherein said tocopherol composition comprises at least about 90% gamma-tocopherol.

8. The medicament of claim 1, wherein said tocopherol metabolite is a metabolite of gamma tocopherol, beta tocopherol or delta tocopherol.

9. The medicament of claim 8, wherein said metabolite is gamma-carboxy ethyl hydroxy chroman (gamma-CEHC).

10. The medicament of claim 1, wherein said tocopherol derivative is a tocotrienol.

11. The medicament of claim 1, wherein said omega-3 poly-unsaturated fatty acid is selected from the group consisting of docosahexaenoic acid (DHA), docosapentaenoic acid (DPA), eicosapentaenoic acid (EPA), eicosatetraenoic acid (ETA), octadecatetraenoic acid, (SDA), and octadecatrientoic acid (ALA).

12. The medicament of claim 11, which contains less than about 10% of an omega-6 poly-unsaturated fatty acid.

13. The medicament of claim 11, wherein said omega-3 poly-unsaturated fatty acid is DHA.

14. The medicament of claim 13, wherein said DHA comprises a ratio of greater than 10:1 DHA:EPA.

15. The medicament of claim 1, which further includes a flavonoid compound.

16. The medicament of claim 15, wherein said flavonoid is selected from the group consisting of quercetin, hesperetin and a mixture of quercetin and hesperetin.

17. The medicament of claim 1, which further comprises a mineral compound.

18. The medicament of claim 17, wherein said mineral compound is selected from the group consisting of copper, zinc, selenium, magnesium, calcium, molybdenum, manganese, chromium, iodine, iron and combinations thereof.

19. The medicament of claim 17, wherein said mineral compound is a divalent ion.

20. The medicament of claim 19, wherein said mineral compound is magnesium.

21. The medicament of claim 1, which further comprises a flavonoid compound and a mineral compound.

22. The medicament of claim 21, wherein said tocopherol composition is a gamma-tocopherol enriched tocopherol composition consisting of greater than about 60% gamma tocopherol, said omega-3 polyunsaturated fatty acid is DHA, said flavonoid is a mixture of hesperetin and quercetin, and said mineral is magnesium.

23. The medicament of claim 22, comprising 100-500 mg of a gamma-tocopherol enriched tocopherol composition, 100-1500 mg DHA, 10-500 mg quercetin, 10-500 mg hesperetin, and 10-500 mg magnesium.

24. The medicament of claim 23, comprising about 300 mg of a gamma-tocopherol-enriched tocopherol composition consisting of at least 60% gamma-tocopherol, about 10% alpha-tocopherol, and about 30% delta-tocopherol; about 800 mg DHA; about 33 mg quercetin; about 66 mg hesperetin; and about 100 mg magnesium.

25. The medicament of claim 1, wherein said medicament is contained in capsular or tablet form.

26. The medicament of claim 25, wherein said tablet or capsular form comprises a plurality of capsules or tablets.

27. The medicament of claim 25, wherein said medicament further comprises a flavonoid compound.

28. The medicament of claim 25, wherein said medicament further comprises a mineral compound.

29. The medicament of claims 1, wherein said medicament is contained in an edible or potable nutritional product.

30. The medicament of claim 29, wherein said nutritional product further comprises a flavonoid compound.

31. The medicament of claim 29, wherein said nutritional product further comprises a mineral compound.

32. The medicament of claim 1, wherein said inflammatory symptoms are associated with PMS, PMDD, perimenopause or menopause.

33. The medicament of claim 32, which further includes a flavonoid compound.

34. The medicament of claim 32, which further includes a mineral compound.

35. The medicament of claim 32, wherein said inflammatory symptoms are selected from the group consisting of acne, bloating, edema, weight gain, breast tenderness, dizziness, dysmenorrhea, fatigue, headache, hot flashes, nausea, diarrhea, constipation, palpitations, swellings of appendages, swelling of breasts, angry outbursts, violent tendencies, anxiety, tension, nervousness, difficulty concentrating, crying easily, depression, food cravings (sweets, salts), forgetfulness, irritability, increased appetite, mood swings, overly sensitive, desire to be alone, abdominal cramps, and backache.

36. The medicament of claim 35, wherein said inflammatory symptoms are selected from the group consisting of bloating, edema and weight gain.

37. The medicament of claim 1, wherein said inflammatory symptoms are associated with concomitant administration of a hormonal contraceptive.

38. The medicament of claim 37, wherein said hormonal contraceptive is an oral contraceptive.

39. The medicament of claim 37, which further includes a flavonoid compound.

40. The medicament of claim 37, which further includes a mineral compound.

41. The medicament of claim 1, wherein said inflammatory biomarker is white blood cell count (WBC).

42. The medicament of claim 1, wherein said inflammatory biomarker is C-reactive protein (CRP).

43. A kit comprising a medicament comprising a non-alpha tocopherol or tocopherol metabolite composition, an omega-3 poly-unsaturated fatty acid, optionally a flavonoid compound and optionally a mineral compound, wherein the components of said formulation are present in a plurality of tablet or capsule forms packaged in separate containers.

44. The kit of claim 43, wherein said kit further includes instructions for determining levels of WBC and/or CRP.

45. The kit of claim 44, wherein said kit further includes measurement means for determining levels of WBC and/or CRP.

46. A medicament for ameliorating or reducing inflammatory symptoms associated with PMS, PMDD, perimenopause or concomitant hormonal contraceptive use in a female mammalian subject, comprising a stoichiometric amount of a tocopherol or tocopherol derivative composition and an omega-9 poly-unsaturated fatty acid, wherein said tocopherol or tocopherol derivative composition and said omega-9 poly-unsaturated fatty acid are present in an amount effective to reduce an inflammatory biomarker in said subject.

47. The medicament of claim 46, wherein said tocopherol composition comprises at least 60% gamma tocopherol and less than about 10% alpha tocopherol, said omega-9 poly-unsaturated fatty acid is all cis 5,8,11 eicosatrienoic acid.

48. The medicament of claim 46, which further comprises a flavonoid.

49. The medicament of claim 48, wherein said flavonoid is selected from the group consisting of quercetin, hesperetin and a mixture of quercetin and hesperetin.

50. The medicament of claim 46, which further comprises a mineral.

51. The medicament of claim 50, wherein said mineral is magnesium.

52. The medicament of claim 46, which further comprises a flavonoid and a mineral.

53. The medicament of claim 46, wherein said inflammatory biomarker is selected from the group consisting of WBC and CRP.

54. A method of ameliorating or reducing one or more premenstrual symptoms in a female mammalian subject experiencing such symptoms or at risk for experiencing such symptoms, comprising administering to the subject a medicament comprising a stoichiometric amount of a non-alpha tocopherol or tocopherol metabolite, and an omega-3 poly-unsaturated fatty acid.

55. The method of claim 54, wherein said symptoms are selected from the group consisting of acne, bloating, edema, weight gain, breast tenderness, dizziness, dysmenorrhea, fatigue, headache, hot flashes, nausea, diarrhea, constipation, palpitations, swellings of appendages, swelling of breasts, angry outbursts, violent tendencies, anxiety, tension, nervousness, difficulty concentrating, crying easily, depression, food cravings, forgetfulness, irritability, increased appetite, mood swings, overly sensitive, desire to be alone, abdominal cramps, and backache.

56. The method of claim 54, wherein the subject is a human subject.

57. The method of claim 54, wherein said female human subject experiences one or more of said symptoms during luteal phase of her menstrual cycle.

58. The method of claim 54, wherein said symptom is dysmenorrhea occurring during late luteal phase or after onset of menstruation.

59. The method of claim 54, wherein said medicament further comprises a flavonoid compound.

60. The method of claim 54, wherein said medicament further comprises a mineral compound.

61. A method of reducing body fluid retention in a mammalian subject, comprising administering to the subject a medicament comprising a stoichiometric amount of a non-alpha tocopherol or tocopherol metabolite, and an omega-3 poly-unsaturated fatty acid.

62. The method of claim 61, wheren said medicament further comprises a flavonoid compound.

63. The method of claim 61, wheren said medicament further comprises a mineral compound.

64. The method of claim 61, wherein the subject is a female human subject.

65. The method of claim 64, wherein said female human subject is in the luteal phase of her menstrual cycle.

66. A method of reducing premenstrual weight gain in a female mammalian subject, comprising administering to the subject a comprising a stoichiometric amount of a non-alpha tocopherol or tocopherol metabolite, and an omega-3 poly-unsaturated fatty acid.

67. The method of claim 66, wheren said medicament further comprises a flavonoid compound.

68. The method of claim 66, wheren said medicament further comprises a mineral compound.

69. The method of claim 66, wherein said subject is a human female subject.

70. The method of claim 69, wherein said weight gain occurs in luteal phase in said subject.

71. A method of reducing the amount of analgesic and/or anti-inflammatory medication required to reduce premenstrual symptoms in a female subject, comprising administering to the subject an effective amount of a medicament comprising a stoichiometric amount of a non-alpha tocopherol or tocopherol metabolite, and an omega-3 poly-unsaturated fatty acid.

72. The method of claim 71, wherein said medicament further comprises a flavonoid compound.

73. The method of claim 71, wherein said medicament further comprises a mineral compound.

74. The method of claim 71, wherein said subject is suffering from PMS, PMDD or perimenopause.
